(19) United States
(12) Patent Application Publication
(10) Pub. No.: US 2013/0129710 A1
(43) Pub. Date: May 23, 2013

(54) HEMOSTATIC COMPOSITIONS

(71) Applicants: Baxter International Inc., Deerfield, IL (US); Baxter Healthcare S.A., Glattpark (Opfikon) (CH)

(72) Inventors: Mark A. Nordhaus, Antioch, IL (US); Paul J. Sanders, Greendale, WI (US); Prasad Dande, Grayslake, IL (US); Shane Donovan, Chicago, IL (US)

(73) Assignees: Baxter Healthcare S.A., Glattpark (Opfikon) (CH); Baxter International Inc., Deerfield, IL (US)

(21) Appl. No.: 13/660,487

(22) Filed: Oct. 25, 2012

Related U.S. Application Data
(60) Provisional application No. 61/552,323, filed on Oct. 27, 2011.

(51) Int. Cl. C07K 14/78

(52) U.S. Cl. CPC C07K 14/78
USPC 424/94.64; 530/354; 536/55.1; 536/51; 536/20; 514/17.2; 525/384; 525/540; 525/420; 525/435

(57) ABSTRACT

The invention discloses a method for producing a hemostatic composition comprising mixing a biocompatible polymer suitable for use in hemostasis and a genipin-type crosslinker, crosslinking said polymer by said genipin-type crosslinker to obtain a crosslinked biocompatible polymer, and finishing said crosslinks polymer to a pharmaceutically acceptable hemostatic composition, new hemostatic compositions and methods for using such compositions.

### CROSS-REFERENCES TO RELATED APPLICATIONS

This application claims the benefit of 61/552,323, filed Oct. 27, 2011, which is incorporated herein by reference.

### FIELD OF THE INVENTION

The present invention relates to hemostatic compositions and processes for making such compositions.

### BACKGROUND OF THE INVENTION

Hemostatic compositions that comprise biocompatible, biodegradable, stable materials are known e.g. from WO98/008550A or WO2003/007845A.

Since such products have to be applied to humans, it is necessary to provide highest safety standards tor quality, storage-stability, and sterility of the final products and the components thereof. In addition, manufacturing and handling should be made as convenient and efficient as possible.

A very successful product in this field utilizes a glutaraldehyde-crosslinked gelatin matrix used either alone or in conjunction with a reconstituted lyophilized thrombin solution, Crosslinking of gelatin or other biomaterial by glutaraldehyde requires careful removal and/or inactivation of unreacted crosslinker before administration to a patient. Various alternative crosslinkers tor biomedical materials have been suggested in the prior art to provide such materials with desired individual characteristics. However, it is usually very difficult to a priori predict the properties and hemostatic performance of a given material after crosslinking with various crosslinking candidate molecules.

It is an object of the present invention to provide a hemostatic composition based on a crosslinked biomaterial wherein the use of glutaraldehyde for crosslinking is avoided. The compositions should also be provided in a convenient and usable manner, preferably as a flowable paste. The products should preferably be provided in product formats enabling a convenient provision of “ready-to-use” hemostatic compositions, which can be directly applied to an injury without any time consuming reconstitution steps.

### BRIEF SUMMARY OF THE INVENTION

Therefore, the present invention provides a new method for producing a hemostatic composition comprising mixing a biocompatible polymer suitable for use in hemostasis and a genipin-type crosslinker, crosslinking said polymer by said genipin-type crosslinker to obtain a crosslinked biocompatible polymer, and finishing said crosslinked polymer to a pharmaceutically acceptable hemostatic composition.

The invention also refers to hemostatic composition comprising a crosslinked biocompatible polymer obtainable by a method according to the present invention, methods of treating an injury or trauma or surgical intervention for example a wound, a hemorrhage, damaged tissue and/or bleeding tissue comprising administering such a hemostatic composition and kits for the treatment of such injury.

Further, a method for providing a ready to use form of a hemostatic composition according to the present invention is disclosed as well as a ready to use hemostatic composition comprising a crosslinked biocompatible polymer obtainable by a method according to the present invention.

### BRIEF DESCRIPTION OF THE DRAWINGS

FIG. 1 shows a schematic representation of the genipin reaction with amino acids (primarily primary amines of lysines) to form intra-molecular protein crosslinks.

FIG. 2 shows TEG Profiles of Glu-Gel and Gen-Gel Variants.

FIGS. 3a and 3b show TEG and % Equilibrium Swell, respectively, with 1 mM genipin.

FIGS. 4a and 4b show TEG and % Equilibrium Swell, respectively, with 2.5 mM genipin.

FIGS. 5a and 5b show TEG and % Equilibrium Swell respectively, with 5 mM genipin.

FIG. 6 shows the evaluation of bleeding severity post test article application and approximation.

FIG. 7 shows the hemostatic success of 5 mM Genipin-Gelatin (27888-51A) in a Porcine Liver Punch-Biopsy Model

FIG. 8a shows Gen-Gel reconstituted and applied upon first application.

FIG. 8b shows Gen-Gel reconstituted and applied after irrigation of excess material.

FIG. 9 shows reconstituted H₂O₂ quenched Gen-Gel variants.

FIGS. 10a and 10b show hemostatic success of Gen-Gel and H₂O₂ quenched Gen-Gel in a Porcine Liver-Punch Biopsy Model.

FIG. 11 shows hemostatic success of a Gen-Gel in a Porcine Liver Punch Biopsy Model.

### DETAILED DESCRIPTION OF THE INVENTION

The present invention provides a method for producing a hemostatic composition comprising mixing a biocompatible polymer suitable for use in hemostasis and a genipin-type crosslinker, crosslinking said polymer by said genipin-type crosslinker to obtain a crosslinked biocompatible polymer, and finishing said crosslinked polymer to a pharmaceutically acceptable hemostatic composition.

Genipin-crosslinked biological material, especially genipin-crosslinked gelatin is known per se (see e.g. U.S. Pat. No. 6,608,040B1, EP2181722A2, WO2008/076407A2, Bigi et al., Biomaterials 23 (2002), 4827-4832; Yao et al., Mat. Chem. Phys. 83 (2004), 204-208; Turo et al., Int. J. Biol. Macromol. (2011), doi: 10.1016; Chiono et al., J. Materl. Sci.:Mater Med. (2008) 19:889-898). With the present invention a flowable hemostatic composition is provided which avoids the use of glutaraldehyde as crosslinking agent. Surprisingly, by using genipin-type crosslinkers, hemostatic materials (“genipin-crosslinked”) can be provided which have comparable or even superior properties compared to glutaraldehyde-crosslinked material. With the present invention, a flowable composition of the material produced can be effectively applied for the treatment of injuries and/or trauma where rapid homeostasis is desired.

The genipin-crosslinked biocompatible polymers according to the present invention, especially genipin-crosslinked gelatin (“Gen-Gel”), have specific advantages over the glutaraldehyde crosslinked materials, especially glutaraldehyde-crosslinked gelatin (“Glu-Gel”), which can be summarized as follows:

As determined by thromboelastography (TEG), the in vitro time to hemostasis is markedly reduced during the first 2 minutes (especially in the first 40 s/the first minute) of the reaction compared to Glu-Gel. Since this reduces blood loss, it is easier to visualize the surgical field, and also to reduce the likelihood of blood transfusions which are themselves associated with poor clinical outcomes. Furthermore, there is increased clot strength using Gen-Gel compared to Glu-Gel. The present Gen-Gel products allow a reduced requirement for the surgeon to approximate the preparation to achieve hemostasis or a faster time to hemostasis, it has improved biocompatibility over glutaraldehyde cross linked based preparations and it can be prepared by a simpler manufacturing process. Moreover, the Gen-Gel products allow a better visualization of the product in the surgical setting (whether by a blue colored variant or a partially decolorized variant compared to gelatin based hemostats), if needed.

Gen-Gel variants (based upon extrusion force data) are similar to or better than the existing Glu-Gel material which facilitates their use in a wider variety of surgical applications. Gen-Gel is comparable to or better than the Glu-Gel product.

A Glu-Gel product has a tendency to be camouflaged by surrounding tissue, since it's slightly yellow color blends in with it. This makes visual evaluation of the desired application problematic. The genipin crosslinked gelatin products according to the present invention appear variable in color from pale yellow to dark blue or green based upon degree of crosslinking reaction conditions, and subsequent processing and finishing steps. This tunabliity of color and ability to obtain desired color in the finished product has the added advantage of providing physicians visual indication of proper product application in wound sites, since this color differentiates it from surrounding tissue, instead of potentially being camouflaged by it. This is another novel feature of this invention. On the other hand, the color can be removed to obtain an essentially non-colored product, depending on the needs with respect to the final products.

Production cost is less for a genipin crosslinked gelatin product according to the present invention than a glutaraldehyde crosslinked one, since reagent, energy, and time costs are lower. The genipin crosslinked gelatin reaction can be performed in water at neutral pH at room temperature for ≦16 hours. The product can be cleaned-up by an ethanol and/or water wash which is not only cheaper, but more importantly, safer for the operator.

The method preferably applies the biocompatible polymer suitable for use in homeostasis as being present in dry form before the crosslinking step.

The preferred genipin-type crosslinker according to the present invention is, of course, genipin (Methyl (1R, 2R, 6S)-2-hydroxy-9-(hydroxymethyl)-3-[4.3.0]nona-4,8-diene-5-carboxylate); however, also other crosslinkers of the iridoid- or secoiridoid-type may be used, such as oleuropein. Preferred concentrations of genipin for crosslinking are in the range of 0.5 to 20 mM, preferably 1 to 1.5 mM, especially 2 to 10 mM.

The biocompatible polymer suitable for use in hemostasis preferably is a protein, a polysaccharide comprising amino groups, a biologic polymer comprising amino groups, a non-biologic polymer comprising amino groups; and derivatives and combinations thereof. Polymers of natural or synthetic origin having nucleophilic groups and/or hydrogen-bond donors/acceptors may also be used. Preferred proteins are selected from the group consisting of gelatin, collagen, albumin, hemoglobin, fibrinogen, fibrin, casein, fibronectin, elastin, keratin, and laminin; and derivatives and combinations thereof. Especially preferred proteins are selected from the group consisting of gelatin, collagen, fibrinogen, fibronectin and fibrin, more preferred gelatin or collagen, especially preferred is gelatin. Preferred polysaccharides comprising amino groups are selected from the group consisting of glycosaminoglycans, pectins, modified starch comprising amino groups, modified cellulose comprising amino groups, modified dextran comprising amino groups, modified hemicellulose comprising amino groups, modified xylan comprising amino groups, modified agarose comprising amino groups, modified alginate comprising amino groups, chitin and chitosan; and derivatives and combinations thereof. Preferred polymers are selected from the group consisting of polyacrylamides, polymethacrylamides, polyethyleneimines, polylysine, polyarginine and polyamidoamine (PAMAM) dendrimers.

According to a preferred embodiment of the present invention, the crosslinked biocompatible polymer is subjected to a quenching/oxidation step with oxidizing agents such as bleach, tBu-hydroperoxide, etc., preferably to a treatment with sodium percarbonate, sodium hypochlorite, chlorine water or hydrogen peroxide (H₂O₂), especially preferred is a treatment with sodium percarbonate or H₂O₂, most preferred is a treatment with percarbonate.

Preferred H₂O₂ concentrations are 0.5 to 20% (w/w), especially 1 to 15% (w/w), more preferred about 5% (w/w). In an especially preferred embodiment the genipin concentration is between 5 to 10 mM, the reaction time of gelatin with genipin is between 3 to 10 hours, especially 6 hours, the H₂O₂ concentration is between 3 to 5% (w/w) and the reaction time of the genipin-crosslinked gelatin with H₂O₂ is about 20 hours.

Preferred sodium percarbonate concentrations are between 1 to 10% (w/w), especially 1 to 5% (w/w), more preferred 1 to 4 % (w/w). In an especially preferred embodiment the genipin concentration is between 5 to 10 mM (especially about 8 nM), the reaction time of gelatin with genipin is between 3 to 10 hours (especially about 5 hours), the sodium percarbonate concentration is between 1 to 10% (w/w), especially preferred between 1 to 4% w/w, and the reaction time of the genipin-crosslinked gelatin with sodium percarbonate is between 1 to 20 hours, preferably between 1 to 5 hours (e.g. 1, 2 or 3 hours).

Quenching may also be carried out in presence of antioxidants such as sodium aseorbate or by controlling oxidation potential of the reaction environment such as carrying out quenching and/or genipin reaction in an inert atmosphere such as nitrogen or argon.

Preferred crosslinking reaction conditions include the performance in aqueous solution, preferably in a phosphate buffered saline (PBS)/ethanol buffer, especially at a pH of 4 to 12, preferably of 5.0 to 10.0, especially of 6 to 8, or in de-ionized water or other aqueous buffers which may contain between 0 to 50% of a water miscible organic solvent. A PBS buffers contains physiological amounts of NaCl and KCl in a phosphate buffer at a physiological pH. An example for a PBS buffer contains 137 mM NaCl, 2.7 mM KCl, 10 mM Na₂HPO₄•2 H₁O, 1.76 mM KH₂PO₄ (pH=7.4). Another example of a PBS buffer consists of 137 mM NaCl, 2.7 mM KCl, 4.3 mM Na₂HPO₄ and 1.4 mM KH₂PO₄ (pH=7.5).

The reaction may also be carried out in an aqueous butler containing up to 50% of a water-miscible organic solvent and/or processing aids such as PEG, PVP, mannitol, sodium percarbortate, sodium lactate, sodium citrate, sodium ascorbate etc.

Preferably, the crosslinking step is performed at a temperature of 4° C. to 45° C., preferably of 15% to 45° C., especially of 20° C. to 40° C.

The crosslinking step may be followed by a quenching step, especially with an amino group containing quencher, preferably an amino acid, especially glycine. With the quencher, yet unreacted genipin-type crosslinkers are inactivated (e.g. by reaction with the quencher in excess) to prevent further crosslinking. Quenching may also be carried out by raising pH of solution to between 8 to 14, or by using nucleophilic compounds containing amino, thiol, or hydroxyl groups and also a combination of pH raising and using nucelophilic compounds. The quenching step after the genipin-gelatin crosslinking reaction according to the present invention, can be actively directed to impart desired physical performance such as swell and TEG which are important determinants of hemostatic activity above and beyond the general genipin-crosslinking alone.

The crosslinked biocompatible polymer is preferably washed after the crosslinking step, preferably by methanol, ethanol or water, especially by de-ionixed water. Another preferred washing step applies an aqueous buffer containing up to 50% (v/v) of water-miscible organic solvent and/or one or more processing aids.

According to a preferred embodiment, the crosslinked biocompatible polymer is dried. In such a dried state, the hemostatic composition is storage-stable for long time even at elevated temperatures (e.g. more than 20° C., more than 30° C. or even more than 40° C.). Preferred dryness conditions include crosslinked biocompatible polymers which are dried to have a moisture content of below 15% (w/w), preferably below 10%, more preferred below 5%, especially below 1%. In another preferred embodiment the product may be supplied in a hydrated or wet state where the hydrating solution may be a biocompatible buffer or solution.

According to a preferred embodiment of the present invention, the biocompatible polymer suitable for use in hemostasis is gelatin, especially type B gelatin. Examples of suitable gelatin materials are described i.a. in examples 1 and 2 of EP1803417B1 and example 14 of U.S. Pat. No. 6,066,325A and U.S. Pat. No. 6,063,061A. Preferably, the biocompatible polymer suitable for use in hemostasis is a gelatin with a Bloom strength of 200 to 400, especially a type B gelatin with a Bloom strength of 200 to 400. Bloom is a test to measure the strength of gelatin. The test determines the weight (in grams) needed by a probe (normally with a diameter of 0.5 inch) to deflect the surface of the gel 4 mm without breaking it. The result is expressed in Bloom (grades). To perform the Bloom test on gelatin, a 6.67% gelatin solution is kept for 17-18 hours at 10° C. prior to being tested.

Gelatin may also be used with, processing aids, such as PVP, PEG and/or dextran as re-hydration aids. In one particular aspect of the present invention, compositions will comprise crosslinked gelatin powders having a moisture content of 20% (w/w) or less, wherein the powder was crosslinked in the presence of a rehydration aid so that the powder has au aqueous re-hydration rate which is at least 5% higher than the re-hydration rate of a similar powder prepared without the rehydration aid. The “re-hydration rate” is defined according to EP 1803417B1 to mean the quantity of an aqueous solution, typically 0.9% (w/w) saline, that is absorbed by a gram of the powder (dry weight basis) within thirty seconds, expressed as g/g: The rehydration rate is measured by mixing the crosslinked gelatin with saline solution for 30 seconds and depositing the wet gelatin on a filter membrane under vacuum to remove the free aqueous solution. One then records the weight of the wet gelatin retained on the filter, dries it (e.g. 2 h at 120° C.), then records the dry weight of the gelatin and calculates the weight of solution that was absorbed per gram of dry gelatin.

Preferred compositions of the present invention will have a re-hydration rate of at least 2 g/g, preferably at least 3.5 g/g, and often 3.75 g/g or higher. Re-hydration rates of similar powders prepared without the re-hydration aids are typically below three, and a percentage increase in re-hydration rate will usually be at least 5%, preferably being at least 10%, and more preferably being at least 25% or higher.

The dry crosslinked gelatin powders of the present invention having improved re-hydration rates are preferably obtained by preparing the powders in the presence of certain re-hydration aids. Such re-hydration aids will be present during the preparation of the powders, but may be removed from the final products. For example, re-hydration aids which are present at about 20% of the total solids content may typically be reduced to below 1% in the final product, often below 0.5% by weight. Exemplary re-hydration aids include polyethylene glycol (PEG), preferably having a molecular weight of between 500 to 20,000; polyvinylpyrrolidone (PVP), preferably having an average molecular weight of up to 50,000; and dextran, typically having an average molecular weight up to 40,000. It is preferred to employ at least two of these rehydration aids when preparing the compositions of the present invention, and more particularly preferred to employ all three. Preferably, the re-hydration aid comprises PEG at from 2.5% to 20% (w/w) based on the weight of the gelatin, PVP at from 1.25% to 20% (w/w), and dextran at from 1.25% to 20% (w/w).

The present invention also refers to new hemostatic composition comprising a crosslinked biocompatible polymer obtainable by a method according to the present invention.

The hemostatic composition according to the present invention preferably comprises a gelatin polymer as crosslinked biocompatible polymer, preferably a type B gelatin polymer. The nature of the gelatin can have advantageous properties on the crosslinking process. Type B gelatin has proven to be specifically advantageous for genipin crosslinking. A specifically preferred gelatin preparation can be prepared by processing young bovine corium with 2 N NaOH for about 1 hour at room temperature, neutralizing to pH 7-8, homogenizing and heating to 70° C. The corium is then fully solubilized to gelatin with 3-10% (w/w), preferably 7-10% (w/w) gelatin in solution. This solution can be cast, dried and ground to provide gelatin type B powder.

The hemostatic composition according to the present invention preferably contains the crosslinked biocompatible polymer in particulate form, especially as granular material. This granular material can rapidly swell when exposed to a fluid (i.e. the pharmaceutically acceptable diluent) and in this swollen form is capable of contributing to a flowable paste that can be applied to a bleeding site. The biocompatible polymer, e.g. gelatin, may be provided as a film which can then be milled to form a granular material. Most of the particles contained in this granular material (e.g. more than 90% w/w) have preferably particle sizes of 10 to 1000 μm, preferably 50 to 800 μm, more preferred 50 to 700 μm, 150 to 700 μm, 200 to 700 μm, especially preferred 300 to 550 μm, most preferred 350 to 550 μm.

According to a preferred embodiment, the biocompatible polymer in particulate form suitable tor use in hemostasis is a crosslinked gelatin. Dry crosslinked gelatin powder can be prepared to re-hydrate rapidly if contacted with a pharmaceutically acceptable diluent. The gelatin granules, especially in the form of a gelatin powder, preferably comprise relatively large particles, also referred to as fragments or sub-units, as described in WO98/08550A and WO2003/007845A. A preferred (median) particle size is 10 to 10000 μm, preferably 50 to 800 μm, more preferred 50 to 700 μm, 150 to 700 μm, 200 to 700 μm, especially preferred 300 to 550 μm most preferred 350 to 550 μm, but particle sizes outside of this preferred range may find use in many circumstances. The dry compositions will also display a significant “equilibrium swell” when exposed to an aqueous re-hydrating medium (=pharmaceutically acceptable diluent, also referred to as reconstitution medium). Preferably, the swell will be in the range from 400% to 1000%. “Equilibrium swell” may be determined by subtracting the dry weight of the gelatin hydrogel powder from its weight when fully hydrated and thus fully swelled. The difference is then divided by the dry weight and multiplied by 100 to give the measure of swelling expressed as percent swell. The dry weight should be measured after exposure of the material to an elevated temperature tor a time sufficient to remove substantially all residual moisture, e.g. after two hours at 120° C. The equilibrium hydration of the material can be achieved by immersing the dry material in a pharmaceutically acceptable diluent, such as aqueous saline, for a time period sufficient for the water content to become constant, typically for from 18 to 24 hours at room temperature.

Exemplary methods for producing crosslinked gelatins are as follows. Gelatin is obtained and suspended in an aqueous solution to form a non-crosslinked hydrogel, typically having a solids content from 1% to 70% by weight, usually from 3% to 10% by weight.

The hemostatic compositions according to the present invention are preferably provided as dry composition, wherein the biocompatible genipin-crosslinked polymer is present in dry form.

A “dry” hemostatic composition according to the present invention has only a residual content of moisture which may approximately correspond to the moisture content of comparable available products, such as glutaraldehyde-crosslinked gelatin (usually have about 12% moisture as a dry product).

The biocompatible polymer in particulate form suitable for use in hemostasis is preferably gelatin in powder form, especially wherein the powder particles have a median particle size of 10 to 1000 μm, preferably 50 to 800 μm, more preferred 50 to 700 μm, 150 to 700 μm, 200 to 700 μm, especially preferred 300 to 550 μm, most preferred 350 to 550 μm. A “dry granular preparation of a biocompatible polymer” according to the present invention is in principle known (yet with different crosslinking) e.g. from WO98/08550A; accordingly, the drying and granulation methods known for e.g. glutaraldehyde-crosslinked gelatin may also be applied for the present genipin-crosslinked material, especially gelatin. Preferably, the polymer is therefore a biocompatible, biodegradable dry stable granular material.

Usually, the polymer particles have a mean particle diameter (“mean particle diameter” is the median size as measured by laser diffractometry; “median size” (or mass median particle diameter) is the particle diameter that divides the frequency distribution in half; fifty percent of the particles of a given preparation have a larger diameter, and 50% of the particles have a smaller diameter) from 10 to 1000 μm, preferably 50 to 800 μm, more preferred 50 to 700 μm, 150 to 700 μm, 200 to 700 μm, especially preferred 300 to 550 μm, most preferred 350 to 550 μm (median size). Although the terms powder and granular (or granulates) are sometimes used to distinguish separate classes of material, powders are defined herein as a special sub-class of granular materials. In particular, powders refer to those granular materials that have the finer grain, sizes, and that therefore have a greater tendency to form dumps when flowing. Granules include coarser granular materials that do not tend to form clumps except when wet.

The present crosslinked biocompatible polymers in particulate form suitable tor use in hemostasis may include dimensionally isotropic or non-isotropic forms, for example, the biocompatible polymers according to the present invention may be granules or fibers; and may be present in discontinuous structures, for example in powder forms.

According to a preferred embodiment, the hemostatic composition is liquid absorbing. For example, upon contact with liquids, e.g. aqueous solutions or suspensions (especially a buffer or blood) the polymer takes up the liquid and will display a degree of swelling, depending on the extent of hydration. The material preferably absorbs from at least 300 %, preferably about 400% to about 2000%, especially from about 500% to about 1300% water or aqueous buffer by weight, corresponding to a nominal increase in diameter or width of an individual particle of subunit in the range from e.g. approximately 50% to approximately 500%, usually from approximately 50% to approximately 250%. For example, if the (dry) granular particles have a preferred size range of 0.01 mm to 1.5 mm, especially of 0.05 mm to 1 mm, the fully hydrated composition (e.g. after administration on a wound or after contact with an aqueous buffer solution) may have a size range of 0.05 mm to 3 mm, especially of 0.25 mm to 1.5 mm.

The equilibrium swell of preferred biocompatible polymers of the present invention may generally range e.g. from 400% to 1300%, preferably being from 500% to 1100%, especially from 600% to 900%, depending on its intended use. Such equilibrium swell may be controlled e.g. (for a crosslinked polymer) by varying the degree of cross-linking, which in turn is achieved by varying the cross-linking conditions, such as the duration of exposure of a cross-linking genipin-type agent, concentration of a cross-linking genipin-type agent, cross-linking temperature, and the like. Materials having differing equilibrium swell values perform differently in different applications. The ability to control crosslinking and equilibrium swell allows the compositions of the present invention to be optimized for a variety of uses. In addition to equilibrium swell, it is also important to control the hydration of the material immediately prior to delivery to a target site. Hydration and equilibrium swell are, of course, intimately connected. A material with 0% hydration will be non-swollen. A material with 100% hydration will be at its equilibrium water content. Hydrations between 0% and 100% will correspond to swelling between the minimum and maximum amounts.

For finishing the crosslinked polymer to a pharmaceutically acceptable hemostatic composition a pharmaceutically acceptable diluent is used.

The pharmaceutically acceptable diluent is preferably an aqueous solution and may contain a substance selected from the group consisting of NaCl, CaCl₂, sodium acetate, sodium lactate, sodium citrate, sodium caprate and mannitol. For example, a pharmaceutically acceptable diluent comprises water for injection, and—independently of each other—50 to 200 mM NaCl (preferably 150 mM), 10 to 80 mM CaCl₂ (preferably 40 mM), 1 to 50 mM sodium acetate (preferably 20 mM) and up to 10% w/w mannitol (preferably 2% w/w). Preferably, the diluent can also include a buffer or buffer system so as to butter the pH of the reconstituted dry composition, preferably at a pH of 3.0 to 10.0, more preferred of 6.4 to 7.5, especially at a pH of 6.9 to 7.1.

According to a preferred embodiment, the pharmaceutically acceptable diluent comprises thrombin, preferably 10 to 1000 I.U. thrombin/ml, especially 250 to 700 I.U. thrombin/ml. Preferably, the hemostatic composition in tins ready to use form contains 10 to 100,000 International Units (I.U.) of thrombin, more preferred 100 to 10,000 I.U., especially 500 to 5,000 I.U. The thrombin concentration in the ready-to-use composition is preferably in the range of 10 to 10,000 I.U., more preferred of 50 to 5,000 I.U.; especially of 100 to 1,000 I.U./ml. The diluent is used in an amount to achieve the desired end-concentration in the ready-to-use composition. The thrombin preparation may contain other useful component, such as ions, buffers, excipients, stabilizers, etc.. Preferably, the thrombin preparation contains human albumin, mannitol or mixtures thereof. Preferred salts are NaCl and/or CaCl₂, both used in the usual amounts and concentrations applied for thrombin (e.g. 0.5 to 1.5 % NaCl (e.g. 0.9%) and/or 20 to 80 mM CaCl₂ (e.g. 40 mM)).

Thrombin (or any other coagulation inducing agent, such as a snake venom, a platelet activator, a thrombin receptor activating peptide and a fibrinogen precipitating agent) can be derived from any thrombin preparation which is suitable for use in humans (i.e. pharmaceutically acceptable). Suitable sources of thrombin include human or bovine blood, plasma or serum (thrombin of other animal sources can be applied if no adverse immune reactions am expected) and thrombin of recombinant origin (e.g. human recombinant thrombin); autologous human thrombin can be preferred for some applications.

The diluent preferably comprises a buffer or butter system, preferably at a pH of 3.0 to 10.0.

preferred embodiment the present invention provides a hemostatic composition comprising genipin-type crosslinked gelatin in particulate form suitable for use in homeostasis, wherein the composition is present in paste form containing a crosslinked biocompatible polymer in an amount of 5 to 30 % (w/w), preferably of 10 to 25% (w/w), especially of 12 to 20% (w/w). in a further embodiment the crosslinked polymer, e.g. gelatin, is present in an amount of 15.0 to 19.5% (w/w) (=weight of dry gelatin per weight of final composition), preferably 16.0 to 19.5% (w/w), 16.5 to 19.5% (w/w), 17.0 to 18.5% (w/w) or 17.5 to 18.5% (w/w), more preferred 16.5 to 19.0% (w/w) or 16.8 to 17.8% (w/w). especially preferred 16.5 to 17.5% (w/w), and wherein the composition optionally comprises an extrusion enhancer, especially albumin. For example, if the extrusion enhancer is albumin (which is specifically preferred, especially human serum albumin), it must be provided in an amount of between 0.5 to 5.0% (w/w)(=weight of extrusion enhancer per weight of final composition), preferably 1.0 to 5.0 % (w/w), preferably 2.0 to 4.5% (w/w)s more preferred 1.5 to 5.0% (w/w), especially preferred about 1.5% (w/w).

In a preferred embodiment the biocompatible polymer, e.g. gelatin, crosslinked with a genipin-type crosslinker, e.g. genipin, is a homogeneously (uniformely) crosslinked polymer as can be shown e.g. by fluorescence measurements as described in Example 6 of the present application. In an especially preferred embodiment the biocompatible polymer, such as gelatin, is present as a homogeneously genipin crosslinked biocompatible polymer, such as gelatin, in particulate form.

According to another aspect, the present invention relates to a hemostatic composition for use in the treatment of an injury selected from the group consisting of a wound, a hemorrhage, damaged tissue, bleeding tissue and/or bone defects.

Another aspect of the present invention is a method of treating an injury selected from the group consisting of a wound, a hemorrhage, damaged tissue and/or bleeding tissue comprising administering a hemostatic composition according to the present invention to the site of injury.

According to another aspect, the present invention also provides a method for delivering a hemostatic composition according to the invention to a target site in a patient's body, said method comprising delivering a hemostatic composition produced by the process according to the present invention to the target site. Although in certain embodiments, also the dry composition can be directly applied to the target site (and. optionally be contacted with the pharmaceutically acceptable diluent a the target site, if necessary), it is preferred to contact the dry hemostatic composition with a pharmaceutically acceptable diluent before administration to the target site, so as to obtain a flowable hemostatic composition in a wetted form, especially a hydrogel form.

In such a method, a kit may be applied, this kit comprising
a) a hemostatic composition according to the present invention; and
b) instructions for use.

A preferred further component of such a kit is—specifically if the hemostatic composition is contained in dry form—a pharmaceutically acceptable diluent for reconstitution of the hemostatic composition. Further components of the kit may be administration means, such as syringes, catheters, brashes, etc, (if the compositions are not already provided in the administration means) or other components necessary for use in medical (surgical) practice, such as substitute needles or catheters, extra vials or further wound cover means, Preferably, the kit according to the present invention comprises a syringe housing the dry and stable hemostatic composition and a syringe containing the diluent (or provided to take up the diluent from another diluent container).

The pharmaceutically acceptable diluent is one as described before.

The diluents may further contain other ingredients, such as excipients. An “excipient” is an inert substance which is added to the solution, e.g. to ensure that thrombin retains its chemical stability and biological activity upon storage (or sterilization (e.g. by irradiation)), or for aesthetic reasons e.g. color. Preferred excipients include proteins e.g. human albumin, carbohydrates, e.g. mannitol, polymers, e.g. polyethylene glycol (PEG) and sodium acetate. Preferred concentrations of human albumin in the reconstituted product are from 0.1 to 100 mg/ml, preferably from 1 to 10 mg/ml. Preferred mannitol concentrations can be in the concentration range of from 0.5 to 500 mg/ml, especially from 10 to 50 mg/ml. Preferred PEG concentrations can be in the concentration range of from 0.5 to 500 mg/ml, especially from 10 to 50 mg/ml. PEG average molecular weights may range from 500 to 20,000. Preferred sodium acetate concentrations are in the range of from 1 to 10 mg/ml, especially 2 to 5 mg/ml.

In a preferred embodiment, the pharmaceutically acceptable diluent is provided in a separate container. This car preferably be a syringe. The diluent in the syringe can then easily be applied to the final container for reconstitution of the dry hemostatic compositions according to the present invention, if the final container is also a syringe, both syringes can be finished together in a pack. It is therefore preferred to provide the dry hemostatic compositions according to the present invention in a syringe which is finished with a diluent syringe with a pharmaceutically acceptable diluent for reconstituting said dry and stable hemostatic composition.

According to a preferred embodiment, the final container further contains an amount of a stabilizer effective to inhibit modification of the polymer when exposed to the sterilizing radiation, preferably ascorbic acid, sodium ascorbate, other salts of ascorbic acid, or an antioxidant.

With such a pharmaceutically acceptable diluent, a ready to use form of the present hemostatic composition may be provided which can then be directly applied to the patient. Accordingly, also method for providing a ready to use form of a hemostatic composition according to the present invention is provided, wherein the hemostatic composition is provided in a first syringe and a diluent for reconstitution is provided in a second syringe, the first and the second syringe are connected to each other, and the fluid is brought into the first syringe to produce a flowable form of the hemostatic composition; and optionally returning the flowable form of the hemostatic composition to the second syringe at least once. Preferably, the ready-to use preparations are present or provided as hydrogels. Products of this kind are known in principle in the art, yet in a different format. Therefore, a method for providing a ready to use form of a hemostatic composition according to the present invention, wherein the hemostatic composition is provided in a first syringe and a diluent for reconstitution is provided in a second syringe, the first and the second syringe are connected to each other, and the diluent is brought into the first syringe to produce a flowable form of the hemostatic composition; and optionally returning the flowable form of the hemostatic composition to the second syringe at least once, is a preferred embodiment of the present invention. This process (also referred to as “swooshing”) provides a suitable ready-to-use form of the compositions according to the present invention which can easily and efficiently be made also within short times, e.g. in emergency situations during surgery. This flowable form of the hemostatic composition provided by such a method is specifically suitable for use in the treatment of an injury selected from the group consisting of a wound, a hemorrhage, damaged tissue, bleeding tissue and/or bone defects.

For stability reasons, such products (as well as the products according to the present invention) are usually provided in a dry form in a final container and brought into the ready-to-use form (which is usually in the form of a (hydro-)gel, suspension or solution) immediately before use, necessitating the addition of wetting or solvation (suspension) agents.

Preferably, the flowable form, of the hemostatic composition contains particles of which more than 50% (w/w) have a size of 100 to 1000 μm, preferably particles of which more than 80% (w/w) have a size of 100 to 1000 μm

Accordingly, the present invention also refers to a ready to use hemostatic composition comprising a crosslinked. biocompatible polymer obtainable by a method according to the present invention. Preferably, the flowable form contains crosslinked biocompatible polymer in an amount of 5 to 30 % (w/w), preferably of 10 to 25% (w/w), especially of 12 to 20% (w/w).

The biocompatible hemostatic crosslinked polymer according to the present invention—once applied to a wound—forms an efficient matrix which can form a barrier for blood flow. Specifically the swelling properties of the hemostatic polymer can make it an effective mechanical harrier against bleeding and re-bleeding processes.

The present composition may additionally contain a hydrophilic polymeric component (also referred to as “reactive hydrophilic component” or “hydrophilic (polymeric) crosslinker”) which further enhances the adhesive properties of the present composition. This hydrophilic polymeric component of the haemostatic composition according to the present invention acts as a hydrophilic crosslinker which is able to react with its reactive groups once the haemostatic composition is applied to a patient (e.g. to a wound of a patient or another place where the patient is In need of a hemostatic activity). Therefore it is important for the present invention that the reactive groups of the polymeric component are reactive when applied to the patient. It is therefore necessary to manufacture the haemostatic composition according to the present invention so that the reactive groups of the polymeric component which should react once they are applied to a wound are retained during the manufacturing process.

For hydrophilic polymeric crosslinkers whose reactive groups which are hydrolysable, premature contact with water or aqueous liquids has to be prevented before administration of the haemostatic composition to the patient, especially during manufacture. However, processing of the hydrophilic polymeric component during manufacturing may be possible also in an aqueous medium at conditions where the reactions of the reactive groups are inhibited (e.g. at a low pit). If the hydrophilic polymeric components can be melted, the melted hydrophilic polymeric components can be sprayed or printed onto the matrix of the biopolymer. it is also possible to mix a dry form (e.g. a powder) of the hydrophilic polymeric component with a dry form of the biocompatible polymer suitable for use in hemostasis, if necessary, then an increase of the temperature can be applied to melt the sprinkled hydrophilic polymeric component to the biocompatible polymer suitable for use in hemostasis to achieve a permanent coating of the hemostatic composition. Alternatively, these hydrophilic polymeric components can be taken up into inert organic solvents (inert vis-à-vis the reactive groups of the hydrophilic polymeric components) and brought onto the matrix of the biomaterial. Examples of such organic solvents are dry ethanol, dry acetone, dry DMF, dioxane, DMSO, or THF (which are e.g. inert for hydrophilic polymeric components, such as NHS-ester substituted PEGs).

In a preferred embodiment the hydrophilic polymer component is a single hydrophilic polymer component and is a polyalkylene oxide polymer, preferably a PEG comprising polymer, The reactive groups of this reactive polymer are preferably electrophilic groups. Alternatively, nucleophilic groups may also be added (e.g. PEG-SH).

The reactive hydrophilic component may be a multi-electrophilic polyalkylene oxide polymer, e.g. a multi-electrophilic PEG. The reactive hydrophilic component can include two or more electrophilic groups, preferably a PEG comprising two or more reactive groups selected from succinimidylesters (—CON((COCH₂)₂), aldehydes (—CHO) and isocyanates (—N=O=O), e.g. a component as disclosed in the WO2008/016983 A (incorporated herein by reference in its entirety).

Preferred electrophilic groups of the hydrophilic polymeric crosslinker according to the present invention are groups reactive to the amino-, carboxy-, thiol- and hydroxy- groups of proteins, or mixtures thereof.

Preferred amino group-specific reactive groups are NHS-ester groups, imidoester groups, aldehyde-groups, carboxy-groups in the presence of carbodiimides, isocyanates, or THPP (beta-[Tris(hydroxymethyl)phosphino] propionic acid), especially preferred is Pentaerythritolpoly(ethyleneglycol)ether tetrasuccinimidyl glutaxate (=Pentaerythritol tetrakis[1- 1-oxo-5′-succinimdylpentanoate-2-poly-oxoethyleneglycole]ether (=an NHS-PEG with MW 10,000).

Preferred carboxy-group specific reactive groups are ammo-groups in the presence of carbodiimides.

Preferred thiol group-specific reactive groups arc maleimides or haloacetyls.

Preferred hydroxy group-specific reactive group is the isocyanate group. The reactive groups on the hydrophilic cross-linker may be identical (homo-functional) or different (hetero-functional). The hydrophilic polymeric component can have two reactive groups (homo/hetero-bi-functional) or more (homo/hetero-trifunctional or more).

In special embodiments the material is a synthetic polymer, preferably comprising PEG. The polymer can be a derivative of PEG comprising active side groups suitable for cross-linking and adherence to a tissue.

By the reactive groups the hydrophilic reactive polymer has the ability to cross-link blood proteins and also tissue surface proteins. Cross-linking to the biomaterial is also possible.

The multi-electrophilic polyalkylene oxide may include two or more succmimidyl groups. The multi-electrophilic polyalkylene oxide may include two or more maleimidyl groups.

Preferably, the multi-electrophilic polyalkylene oxide is a polyethylene glycol or a derivative thereof.

In a most preferred embodiment the hydrophilic polymeric component is pentaerythritolpoly(ethyleneglycol)ether tetrasuccinimidyl glutarate (=COH102, also pentaerythritol tetrakis[1-1′-oxo-5′-succinimidylpentanoate-2-poly-oxoethyleneglycole]ether).

The hydrophilic polymeric component is a hydrophilic crosslinker. According to a preferred embodiment, this crosslinker has more than two reactive groups for crosslinking (“arms”), for example three, four, five, six, seven, eight, or more arms with reactive groups for crosslinking. For example, NHS-PEG-NHS is an effective hydrophilic crosslinker according to the present invention. However, for some embodiments, a 4-arm polymer (e.g. 4-arms-p-NP-PEG) may be more preferred; based on the same rationale, an 8-arm polymer (e.g. 8-arms-NHS-PEG) may even be more preferred for those embodiments where multi-reactive crosslinking is beneficial. Moreover, the hydrophilic crosslinker is a polymer, i.e. a large molecule (macromolecule) composed of repeating structural units which are typically connected by covalent chemical bonds. The hydrophilic polymer component should have a molecular weight of at least 1000 Da (to properly serve as crosslinker in the hemostatic composition according to the present invention); preferably the crosslinking polymers according to the present invention has a molecular weight of at least 5000 Da, especially of at least 8000 Da.

For some hydrophilic crosslinkers, the presence of basic reaction conditions (e.g. at the administration site) is preferred or necessary for functional performance (e.g. for a faster cross-linking reaction at the administration site). For example, carbonate or bicarbonate ions (e.g. as a buffer with a pH of 7.6 or above, preferably of 8.0 or above, especially of 8.3 and above) may be additionally provided at the site of administration (e.g. as a buffer solution or as a fabric or pad soaked with such a buffer), so as to allow an improved performance of the hemostatic composition according to the present invention or to allow efficient use as a hemostatic and/or wound adherent material.

The reactivity of the hydrophilic polymeric component (which, as mentioned, acts as a crosslinker) in the composition according to the present invention is retained in the composition. This means that the reactive groups of the crosslinker have not yet reacted with the haemostatic composition and are not hydrolyzed by water (or at least not in a significant amount which has negative consequences on the hemostatic functionality of the present compositions). This can be achieved by combining the hemostatic polymer with the hydrophilic crosslinker in a way which does not lead to reaction of the reactive groups of the crosslinker with the hemostatic polymer or with water. Usually, this includes the omitting of aqueous conditions (or wetting), especially wetting without the presence of acidic conditions (if crosslinkers are not reactive under acidic conditions). This allows the provision of reactive hemostatic materials.

Preferred ratios of the biocompatible crosslinked polymer to hydrophilic polymeric component in the hemostatic composition according to the present invention are from 0.1 to 50 % w/w, preferably from 5 to 40% w/w.

Further components may be present in the hemostatic composition according to the present invention. According to preferred embodiments, the hemostatic compositions according to the present invention may further comprise a substance selected from the group consisting of antifibrinolytic, procoagulant, platelet activator, antibiotic, vasoconstrictor, dye, growth factors, bone morphogenetic proteins and pain killers.

The hemostatic composition according to the present invention may comprise a further composition of genipin-crosslinked gelatin and a polyvalent nucelophilic substance, preferably human serum, albumin, optionally at a basic pH (e.g. pH 8 to 11, preferably 9 to 10, especially at a pH 1 of 9.5).

The present invention also refers to a brushed final container obtained by die process according to the present invention. This finished container contains the hemostatic composition according to the present invention in a sterile, storage-stable and marketable form. The final container can be any container suitable for housing (and storing) pharmaceutically administrate compounds. Syringes, vials, tubes, etc. can be used; however, providing the hemostatic compositions according to the present invention in a syringe is specifically preferred. Syringes have been a preferred administration means for hemostatic compositions as disclosed in the prior art also because of the handling advantages of syringes in medical practice. The compositions may then preferably be applied (after reconstitution) via specific needles of the syringe or via suitable catheters. The reconstituted hemostatic compositions (which are preferably reconstituted to form a hydrogel) may also be applied by various other means e.g. by a spatula, a brush, a spray, manually by pressure, or by any other conventional technique. Administration of the reconstituted hemostatic composition to a patient by endoscopic (laparoscopic) means is specifically preferred. Usually, the reconstituted hemostatic compositions according to the present invention will be applied using a syringe or similar applicator capable of extruding the reconstituted composition through an orifice, aperture, needle, tube, or other passage to form a bead, layer, or similar portion of material. Mechanical disruption of the compositions can be performed by extrusion through an orifice in the syringe or other applicator, typically having a size in the range from 0.01 mm to 5.0 mm, preferably 0.5 mm to 2.5 mm.

Preferably, however, the hemostatic composition will be initially prepared from a dry form having a desired panicle size (which upon reconstitution, especially by hydration, yields subunits of the requisite size (e.g. hydrogel subunits)) or will be partially or entirely mechanically disrupted to the requisite size prior to a final extrusion or other application step. It is, of course evident, that these mechanical components have to be provided in sterile form (inside and outside) in order to fulfill, safety requirements for human use.

Another aspect of the invention concerns a method for providing a ready-to-use hemostatic composition comprising contacting a hemostatic composition produced by the process according to the present invention with a pharmaceutically acceptable diluent.

The invention is further described in the examples below and the drawing figures, yet without being restricted thereto.

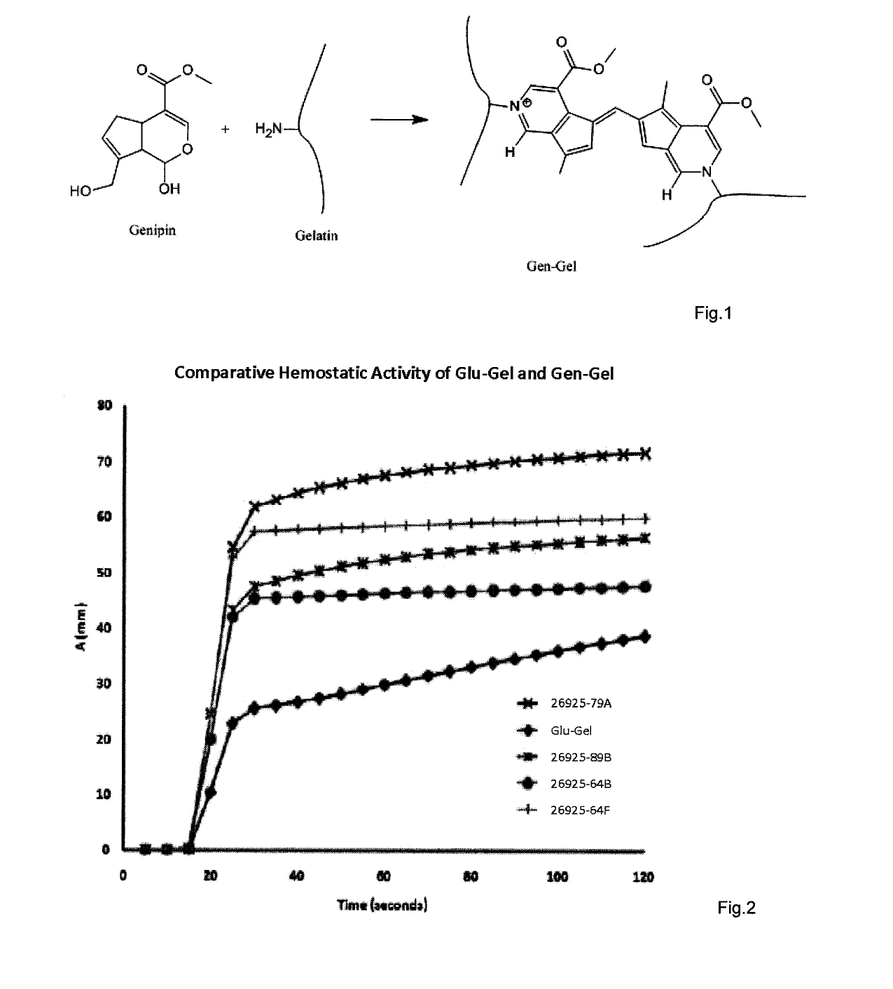

FIG. 1 shows a schematic representation of the genipin reaction with amino acids (primarily primary amines of lysines) to form intra-molecular protein crosslinks.

FIG. 2 shows TEG Profiles of Glu-Gel and Gen-Gel Variants.

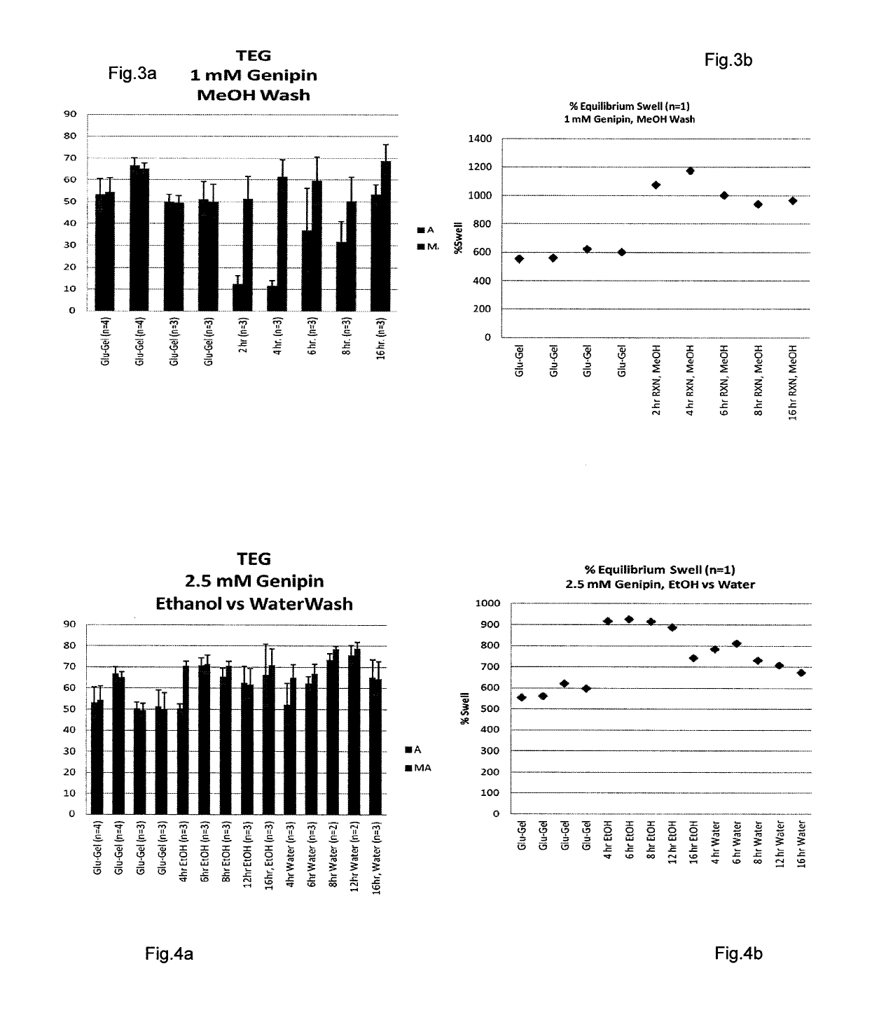

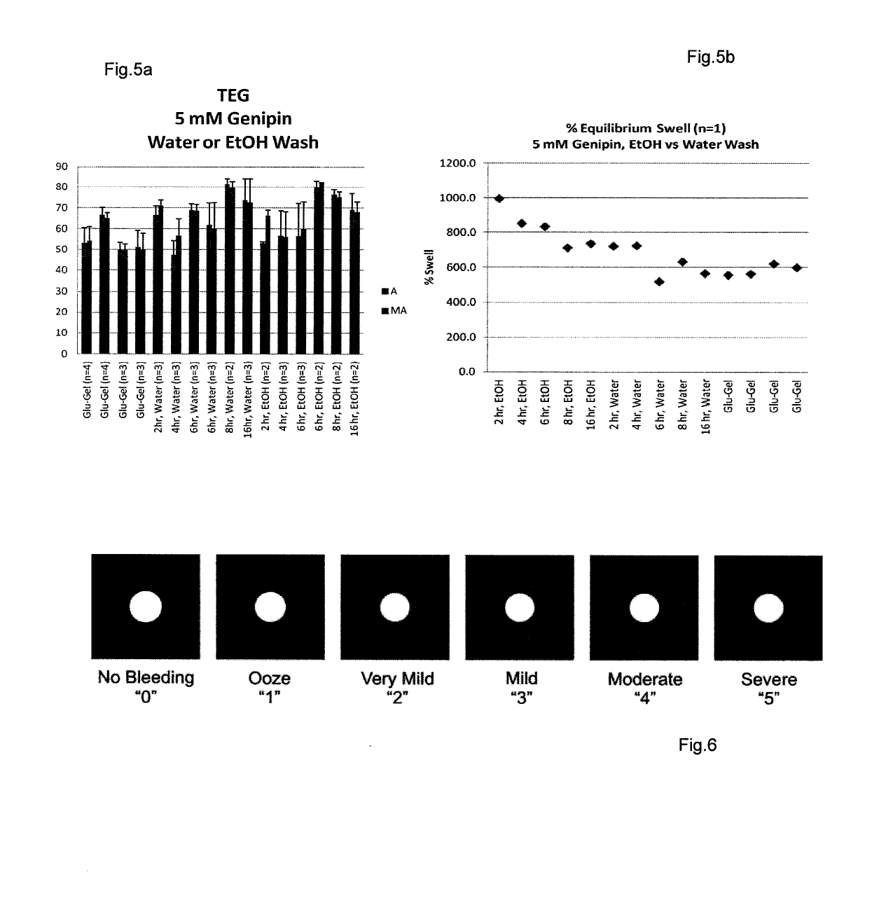

FIGS. 3, 4 and 5 show TEG and % Equilibrium Swell with 1 mM genipin (FIG. 3), 2.5 mM genipin (FIG. 4) and 5 mM genipin (FIG. 5).

FIG. 6 shows evaluation of bleeding severity post test article application and approximation.

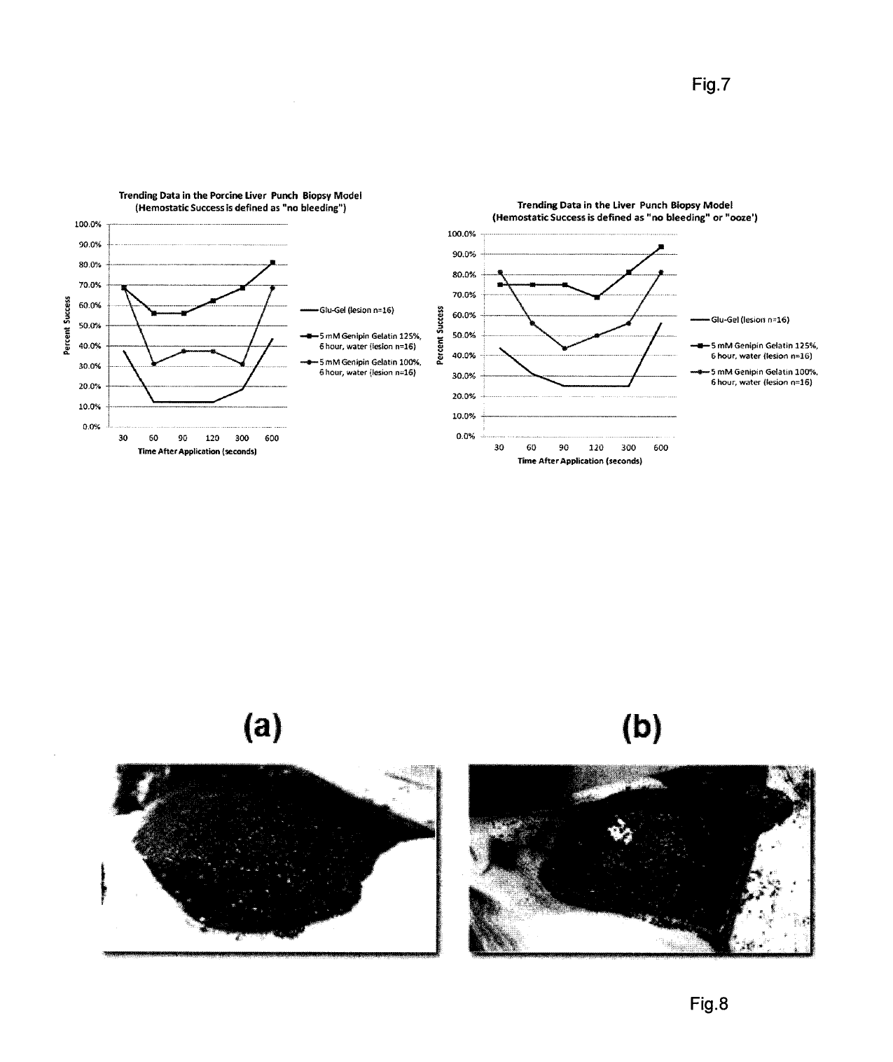

FIG. 7 shows the hemostatic success of 5 mM Genipin-Gelatin (27888-51A) in Porcine Liver Punch-Biopsy Model.

FIG. 8 shows Gen-Gel reconstituted and applied (a) upon first application; (b) after irrigation of excess material.

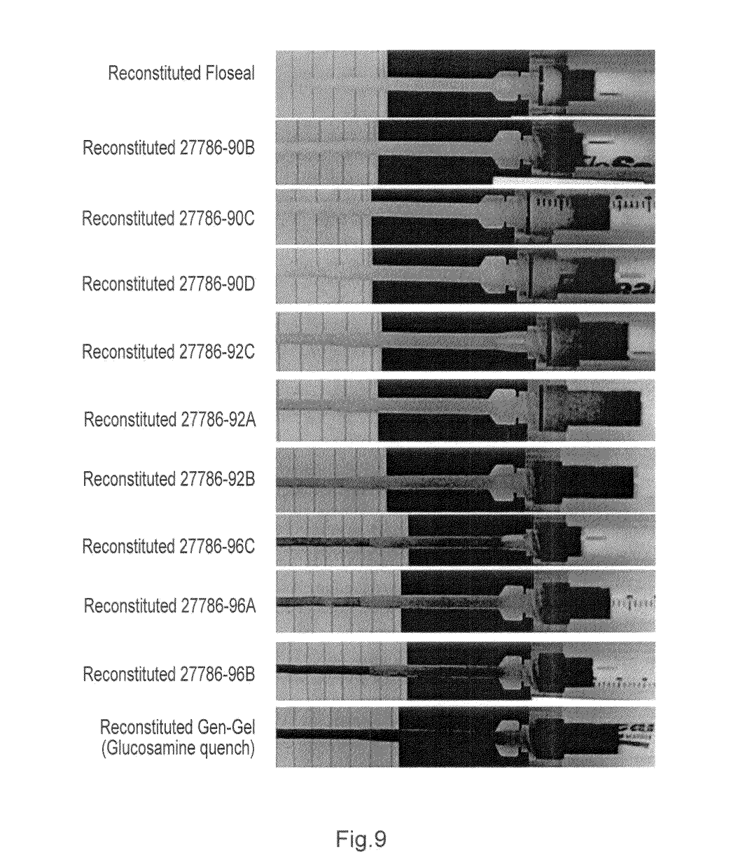

FIG. 9 shows reconstituted H₂O₂ quenched Gen-Gel variants.

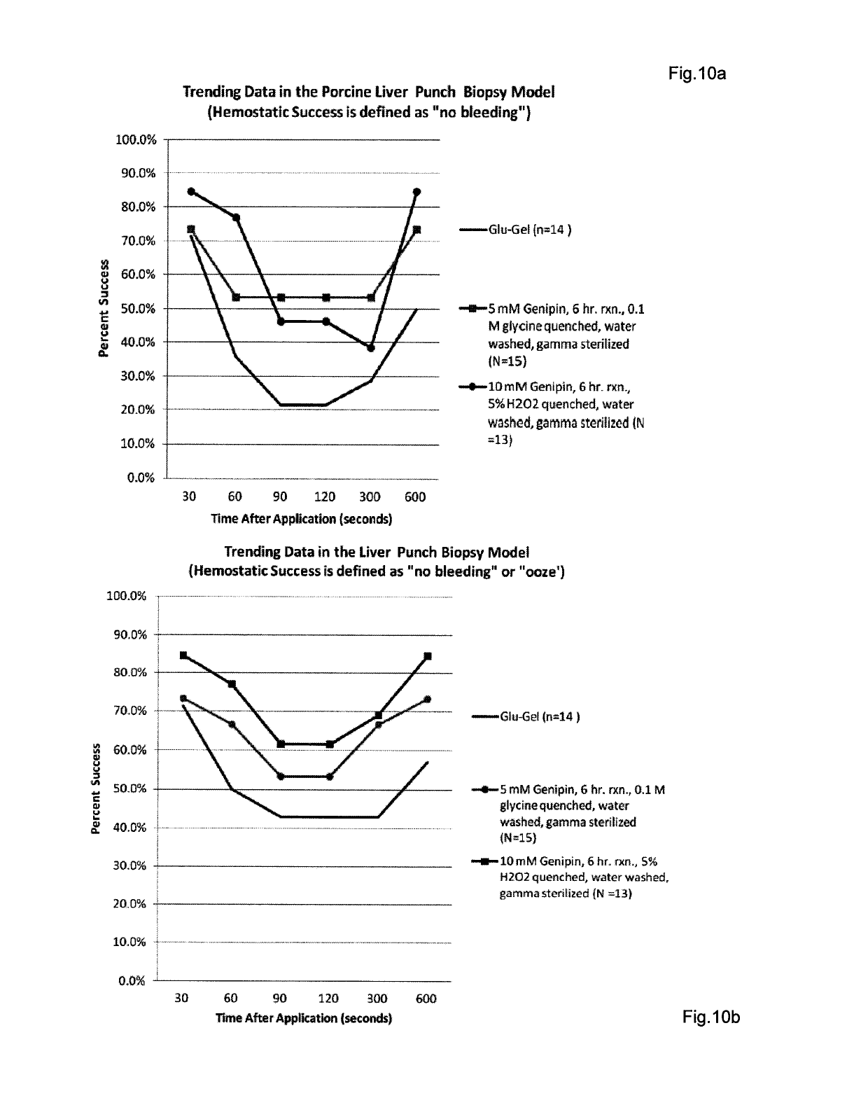

FIG. 10 shows hemostatic success of Gen-Gel and H₂O₂ quenched Gen-Gel in Porcine Liver-Punch Biopsy Model.

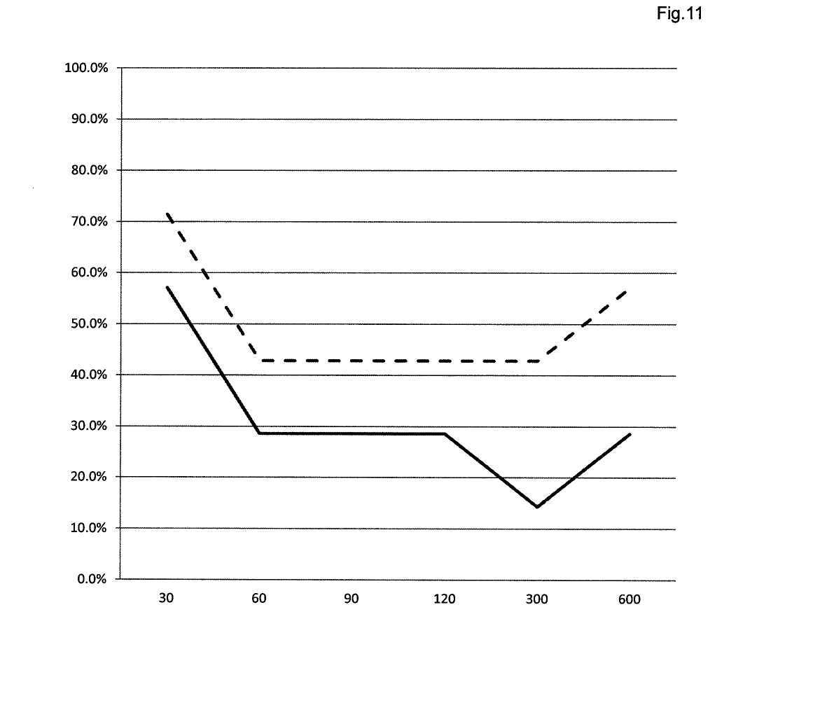

FIG. 11 shows hemostatic success (defined as “no bleeding”) of a Gen-Gel preparation according to Chiono et ah in a Porcine Liver Punch Biopsy Model.
shows a preparation according to the present invention
shows a preparation according to Chiono et al.

The x-axis shows time after application in [seconds], the y-axis shows percent hemostatic success.

The following abbreviations are used:
DIW de-ionized process water
ETOH ethanol
EF extrusion force
hr(s) hour(s)
H₂O water
MeOH methanol
RT room temperature
sec second(s)

### EXAMPLES

Genipin is an aglycone derived from geniposide, which is found in the fruit of Gardenia jasminoides Ellis. Genipin possesses the molecular formula C₁₁H₁₄ O₅ and contains a dihydropyran ring. Genipin reacts spontaneously with amino acids (primarily primary amines of lysines) to form intra-molecular protein crosslinks (FIG. 1). Crosslinked proteins appear as a dark blue in color. Genipin can crosslink primary amines in gelatin, the same functionalities that are crosslinked by glutaraldehyde. This change had minimal impact on manufacturing procedures and performance of final product. Surprisingly, the genipin-crosslinked gelatin product (“Gen-Gel”) has demonstrated some unexpected performance and manufacturing advantages over glutaraldehyde-crosslinked gelatin (“Glu-Gel”) material. These are presented in further detail in the present example section.

### Example 1

### Genipin Crosslinked Gelatin

For proof of concept demonstration a number of Gen-Gel variants were synthesized. Reactions were carried out in PBS at pH 7.4 containing approx, 5% EtOH. Reactions were performed at RT and at 38° C. The gelatin concentration was held constant at 5% w/v. Different concentrations (5 mM, 9.7 mM and 2.5 mM) of genipin were evaluated and reactions were allowed to proceed for either 16 or 39 hrs. Reactions were quenched by either washing exhaustively, first with ETOH and next with H₂O or by suspending in 0.25 M Glycine solution (pH 9.9) for 24 h, followed by exhaustive EtOH/H₂O wash. All reaction products were washed finally with MeOH and dried in oven at 34° C.

The dried reaction products were size-reduced by attrition and sized between sieve #25 & sieve 480 giving a nominal size distribution between 177 μm to 710 μm. Following this final processing step the different variants (Table 1) were analyzed/evaluated by TEG, EF test. Equilibrium swell test, and microscopy.

TABLE 1
Gen-Gel set ups

| | Genipin | Reaction | Reaction | |
|---|---|---|---|---|
| | Conc. | Temperature | Time | |
| | (mM) | (° C.) | (h) | Processing |
| Un-crosslinked | N/A | N/A | N/A | N/A |
| Gelatin | | | | |
| Glu-Gel Matrix | N/A | N/A | N/A | N/A |
| 26925-79A^{rev} | 5.1 | 22 | 17 | Glycine quench |
| 26925-79B^{rev} | 5.1 | 38 | 17 | Glycine quench |
| 26925-89A^{rev} | 5.1 | 22 | 39 | EtOH wash only |
| 26925-89 B^{rev} | 5.1 | 22 | 16 | EtOH wash only |
| 26925-95A | 5.1 | 22 | 39 | Glycine quench |
| 26925-100A | 2.5 | 22 | 16 | EtOH wash only |
| 26925-102A | 2.5 | 22 | 16 | Glycine quench |
| 26925-64B | 9.7 | 22 | 16 | EtOH wash only |
| 26925-64F | 9.7 | 38 | 16 | EtOH wash only |

Gen-Gel variants were found to have similar equilibrium swell (between 400 to 900%), EF (<10 lbf, represent, samples), and comparable or superior TEG performance (FIG. 2) to Glu-Gel. The TEG profiles according to FIG. 2 show that Gen-Gel forms a clot that is at least as fast to form and as strong as floseal VH S/D. In fact, Gen-Gel showed TEG amplitude values of 40 or more within 40 s (some variants even, over 50 or over 60 after 40 s). This behavior was observed over a range of reaction conditions, indicating a robust and tunable synthetic process. The results also show that synthesis is robust and straightforward.

Experiments were repeated by performing the reaction in de-ionized H₂O and similar results were obtained.

### Example 2

### Performance Testing

As In Example 1, three key reaction parameters were systematically adjusted in the further manufacturing experiments. Genipin concentration (1, 2.5 and 5 mM) and reaction time (2, 4, 6, 8, 12 and 16 h) was varied and compared with Glu-Gel. Also post-synthesis steps were varied (e.g. H₂O wash vs. alcohol/H₂O wash).

The effect of systematically adjusting these reaction parameters on product performance (SAR) was evaluated using the following tests:
a) % Equilibrium Swell:

Testing was performed by the following method (also disclosed in example 7 of EP 1803 417 B1): Sample is hydrated in 0.9% saline solution for ˜20 hrs, and its swell weight is determined. Sample is then dried at 120° C. for 2 h and its dry weight is determined. The weight difference is used to calculate % equilibrium swell.
b) Thromboelastography (TEG):

TEG was performed by the following method: For the present examples a TEG® 5000 Thromboelastography® Hemostasis System was used employing software TEG Analytical Software (TAS) Version 4.2.3. In brief, 0.125 g of test article is reconstituted with 625 μl of the thrombin stock solution containing 500 IU/ml thrombin and 40 mMCaCl₂ which is then left to sit for 5 min. Approximately 150 μl or 150 mg of the reconstituted test article is transferred to a TEG cup which is placed into the instrument. Immediately 210 μl of the blood anti-coagulated with 5 U/ml of heparin is added to the cup and quickly mixed. The TEG is then started and collects data for typically 20 minutes. The Amplitude (A) and Maximal Amplitude (MA) values were used to score product performance. Glu-Gel was used as a reference standard. A representative TEG profile for Glu-Gel would provide values such as A=66.2 mm, MA =65.4 mm and A/MA=1.012. A and MA values >50 mm and an A/MA value of >1 are predictors of good hemostatic activity and robust clot formation.
c) Extrusion force (EF):

EF analysis was performed to determines force values for 5 cc syringes with a male Luer lock system (with a cylindric body having an inner diameter of 0.482 inch) having a standard 6.35 cm delivery tip attached to it. In brief 0.80 g test article was transferred into a 5.0 ml Matrix (as described above) syringe. 4.0 ml Thrombin/CaCl₂ stock solution (containing 500 IU/ml thrombin and 40 mM CaCl₂ and approx. 50 mg/ml albumin) was taken-up into a 5.0 ml standard syringe with a female luer lock system. The two syringes were connected and the test article was rapidly reconstituted 20 times and then allowed to wait for 30±3 minutes prior to analysis. The interconnected syringes were then “swooshed” two more times, and the syringe with the male luer lock system containing the reconstituted sample was fitted with the applicator tip and inserted into the MTS Insight™ Electromechanical force gauge. The sample was extruded at a set compression rate of 250 mm/minute, and its mean force determined over total sample extrusion was recorded.

From these forces the mean extrusion forces are calculated as follows:

$$\frac{Total Energy ( mJ )}{Max . Deflection ( mm )} = Mean Force ( N )$$

The maximum extrusion force required was also measured and the allowable upper force limit was set to 10 lbf.

The syringes and the applicator are commercially available as parts of the Floseal Hemostatic Matrix from the company Baxter.
d) Collagenase Assay:

In addition a preliminary in vitro enzymatic (Collagenase) assay was performed on a subset of Gen-Gel variants. The collagenase assay was performed by the following method: 0.08 g of each samples was incubated with 2 ml of PBS puffer for 30 minutes at 37° C. in an end over end mixer. Thereafter the samples were subjected to centrifugation in an Eppendorf centrifuge at 14000 rpm for 5 minutes at RT. The supernatant was discarded and the precipitate was re-suspended in 1.2 ml PBS buffer containing 0.111 U/ml collagenase. A reference sample was incubated with 1.2 ml PBS buffer (without the addition of collagenase). The samples were incubated at 37° C. in an end over end mixer and after defined standing times the supernatants were aspirated, weighed and collected for protein determination (BCA test) and samples were refilled with 1.2 ml of PBS buffer containing collagenase. The time to lysis can be determined by measuring the content of the degraded proteins that were released into the supernatants over time. This assay measures the estimated 90% lysis time of the test article and is an indirect estimate of the potential in vivo residence time of test articles. Glu-Gel was used as a reference standard, and 90% lysis times, which is an indirect estimate of the potential in vivo residence time of test articles, were compared with the values obtained for Glu-Gel.

Results and Discussion

TEG & % Equilibrium Swell Performance

In the first set of reactions the effect of reaction time on product performance was evaluated, when 1 mM genipin. was used in the crosslinking reactions, As expected, performance in terms of both TEG and % Swell improved with Increase in reaction time (FIG. 3a and 3b). The 16 hrs reaction has a TEG performance (FIG. 3a) in the same range as the Glu-Gel reference standards.

In the second set of reactions (FIG. 4) the effect of reaction time, and processing protocols on product performance was evaluated at 2.5 mM genipin reaction concentration. increasing the genipin concentration to 2.5 mM resulted in excellent TEG and % Swell performance (FIG. 4a and 4b) across all reaction times and processing conditions.

In the third set of reactions (FIG. 5) product performance was evaluated at 5 mM genipin reaction concentration. TEG performance (FIG. 5a) across all reaction times and processing conditions was further improved over the 2.5 mM genipin reactions. As expected, performance in terms % Swell also improved. Washing with H₂O once again, produced crosslinked variants with reduced % Swell vs. EtOH washes (FIG. 5b).

Effect of Reaction at Process Conditions on Extrusion Force (EF)

EF directly impacts ease of use. EF measurements were performed on a subset of the Gen-Gel variants synthesized. Representative data is presented in table 2.

TABLE 2
EF values for Representative Gen-Gel Variants

| | Test Article | | |
|---|---|---|---|
| Sample | (Genipin Conc., | EF (lbf) 0.2 g | EF (lbf) 0.25 g |
| Ref. ID's | Reaction Time, Wash) | gelatin/ml | gelatin/ml |
| Floseal | Glu-Gel Matrix | 1.5-1.7 | 2.8-3.8 |
| 26925-184E4-S | 2.5 mM, 16 h, EtOH | 7.9 | 16.9 |
| 27786-72A | 2.5 mM, 4 h, H₂O | 3.0 | 5.0 |
| 27786-71A | 2.5 mM, 6 h, H₂O | 3.5 | 7.6 |
| 27786-72B | 2.5 mM, 16 h, H₂O | 4.0 | 6.6 |
| 27888-9A-S | 5 mM, 6 h, EtOH | 3.9 | N/A |
| 26925-180E2-S | 5 mM, 16 h, EtOH | 10.6 | 17.2 |
| 27786-71B | 5 mM, 6 h, H₂O | 4.4 | 7.5 |
| 27786-58A | 5 mM, 16 h, H₂O | 3.2 | 6.8 |

EF measurements revealed an interesting trend which mirrored the observation from the % Swell measurements. As discussed in the previous section alcohol washing the Gen-Gel product resulted in significantly higher % Swell values as opposed to washing with H₂O. This trend is repealed in the EF measurements where EtOH washed variants have significantly higher EF values compared to the analogous H₂O washed variants.

Indirect in vivo Residence Time Estimation—In vitro Collagenase Assay

The 5 mM genipin series of variants was tested in the in vitro collagenase assay and 90% lysis times were established (table 3). The time for 90% lysis is directly proportional to the reaction time for each variant. Increase in reaction time is expected to result in an increase in degree of crosslinking and consequently require longer exposure to collagenase for 90% lysis. The results support this hypothesis. The 90% lysis time for the 5 mM genipin, 6 hrs reaction, with H₂O wash processing most closely mimics the reference Glu-Gen matrix.

TABLE 3
Collagenase assay performance

| Test Article (Genipin Conc., | Estimated 90% |
|---|---|
| Reaction Time, Wash) | Lysis Time (h) |
| Glu-Gel Matrix | 84 |
| 5 mM, 4 h, H₂O | 68 |
| 5 mM, 6 h, H₂O | 86 |
| 5 mM, 8 h, H₂O | 96 |
| 5 mM, 16 h, H₂O | 160 |

Conclusions

Genipin concentration, reaction time, and the washing process were the parameters evaluated. The Gen-Gel variants generated were evaluated using in vitro performance measures such as TEG, % Swell, EF and collagenase degradation assay. A range of product properties could be obtained by varying reaction and process conditions. This is summarised in table 4 using the 5 mM genipin series as a representative example.

Effect of Genipin Concentration & Reaction Time:

Increasing the genipin reaction concentration and/or increasing the reaction time increases the number of chemical reaction events with gelatin, over a given period of time, which in turn results in an increase in crosslinking density. Hence increase in either one or both of these parameters leads to a lower swell, improved TEG, and lower extrusion force product. Consequently, an increase in 90% lysis time is observed that is directly proportional to both these parameters. Thus, genipin concentration and. reaction time are two key variables for controlling degree of crosslinking and product performance.

Importance of Post Synthetic Processing—The Wash:

Washing with alcohol compared to H₂O results in products that have strikingly different performance profiles. The alcohol wash has an advantage of foster post-processing drying time (20 h) over the H₂O wash (60 hrs). However the H₂O has the dual advantage of being environmentally friendly and also producing a product with better performance in all three performance criteria.

From the chemistry and performance analysis, the 5 mM genipin, 6 hrs reaction, followed by a H₂O wash was selected as the lead candidate to be evaluated in a porcine-diver model.

TABLE 4
Representative Performance Data for 5
mM Genipin Crosslinked Gelatin Variants

| Gen-Gel Variant | | % | | 90% Lysis |
|---|---|---|---|---|
| (Genipin Conc., | | Equilibrium | Extrusion | Time (Relative |
| Reaction Time, Wash) | TEG | Swell | Force | to Glu-Gel) |
| Glu-Gel Matrix | Good | Good | Good | = |
| 5 mM, 2 h, EtOH | OK | OK | ND | < |
| 5 mM, 4 h, EtOH | Good | OK | ND | < |
| 5 mM, 6 h EtOH | Good | OK | OK | < |
| 5 mM, 8 h, EtOH | Good | Good | OK | < |
| 5 mM, 16 h, EtOH | Good | Good | OK | > |
| 5 mM, 2 h, H₂O | OK | Good | ND | ND |
| 5 mM, 4 h, H₂O | OK | Good | ND | < |
| 5 mM, 6 h, H₂O | Good | Good | Good | = |
| 5 mM, 8 h, H₂O | Good | Good | Good | > |
| 5 mM, 16 h, H₂O | Good | Good | Good | > |

### Example 3

### Porcine Liver Punch-Biopsy Model

Materials and Methods:

Animal Model

For this model, a midline laparotomy is performed, followed by electrocautery to stop the bleeding from the surgical incision. The liver is exposed and a lobe is isolated. A 10 mm diameter punch biopsy is used to create a series of 2, non-full thickness lesions, approximately 5 mm deep, with the core tissue removed. A pre-treatment assessment is made on the lesion which includes collecting the blood flowing from each lesion for 10 sec. with pre-weighed gauze.

Test articles are randomized and presented to the surgeon, who is blinded to the sample treatment. Approximately 1.0 ml of the assigned test article is topically applied to a lesion. Saline moistened gauze is used to help approximate the test articles to their designated lesions, and the timer is started. The saline moistened approximation, gauze is removed after 30 seconds.

The degree of bleeding is assessed at 30, 60, 90,120, 300, and 600 sec. after the test articles are applied to their designated lesions as per the depictions in FIG. 6.

Product saturated with blood, but without active bleeding is scored as a “0” (zero). Saline is used to irrigate the excess test articles away from the lesions after the 300 sec. assessment. The procedure is repeated and performed in multiple liver lobes. A single surgeon creates, treats, and performs the observation assessments. The surgeon may also obtain video/photographic data on product performance, appearance, ease of use, etc..

Test article for the in vivo evaluation in the porcine-liver model was synthesized as per the synthetic procedures developed in the molecular design and synthetic chemistry group and detailed in example 2.

0.7918 g of genipin were dissolved in 35 ml absolute EtOH. To this, 665 ml of PBS, pH 7.5 and 35 grams of un-crosslinked gelatin were added with constant stirring and the resulting suspension was stirred at RT for 6 hrs. The color of the suspension changed from a very faint brown to an intense blue over the period of the reaction. After 6 hrs crosslinked genipin-gelatin product was isolated by filtration and washed exhaustively with de-ionized H₂O. It was then re-suspended in 700 ml of a 100 mM glycine solution and stirred overnight at RT. The product was filtered once again and washed with de-ionised H₂O till the washings had a conductivity reading of <10 μS/cm. The filtered product was transferred to a glass baking tray and dried in an oven at 34° C. for approx. 3 days. The dry Gen-Gel product was removed from the oven and ground using a Hamilton-Beach coffee grinder set to “drip” setting. The ground powdered product was sieved between sieve # 25 (mesh size approx=700 microns) and sieve #80 (mesh size approx=180 microns). The product was designated as lot number 27786-86B,

The test article was portioned in 5 ml syringes, (0.8 g/syringe) re-designated as 27888-51A and evaluated in the porcine-liver model.

Test Article Formulation

in-vivo evaluation of 27888-51A. was performed using the porcine liver punch-biopsy model in 2 animals, 1 per day. A thrombin/CaCl₂ stock solution was prepared containing 500 I.U./ml thrombin in a 40 mM CaCl₂ solution. Test article 2788-51A was evaluated in 2 formulations:
1.5 mM Genipin-Gelatin 0.25 g/ml gelatin (27888-51A-125)
2.5 mM Genipin-Gelatin 0.2 g/ml gelatin (27888-51A-100)

Glu-Gen was used as reference standard. Each sample was rapidly mixed by passage between syringes (“swooshed”) 20 times, allowed to rest for 5 minutes, and then re-swooshed 3 more times. 1 ml aliquots of reconstituted test article were dispensed into individual 3 ml volume syringes. These individual 3 ml syringes were then used to apply test article to liver punch lesions at various time points.

Results and Discussion

In-vivo evaluation of 27888-51A was performed using the porcine liver punch-biopsy model in 2 animals, 1 animal per day. The results are presented in FIG. 7. The data presented in FIG. 7 clearly demonstrates the hemostatic success of 27888-51A (27786-86B) in the porcine over punch-biopsy model. 5 mM Gen-Gel synthesized as per the procedure described above leads to hemostatic success in both formulations. Moreover, in this model, the Gen-Gel formulation outperforms the Glu-Gen formulation in terms of percent success.

### Example 4

### Decolorizing Gen-Gel

Gelatin crosslinked with genipin product has a deep blue color. This color is retained upon reconstitution and application of the product at the site of the bleed (FIG. 8). The blue color in the Gen-Gel product is a result of a blue chromophore formed during the reaction of the amine groups with genipin. The product of the genipin-amine reaction has a number of unsaturated (double) bonds in conjugation resulting in absorption of light in the visible spectrum and an intense blue color. According to a preferred embodiment of the present invention, a desired color aside from blue may be introduced into the final Gen-Gel product. This has the advantage of tailoring the color to the desired application including but not limited to hemostasis. Different colors may also be preferred for hemostasis depending on the specific surgical procedure or wound location.

The blue color in Gen-Gel is a direct result of the number of crosslinking reactions between genipin and gelatin. Alteration of the color is therefore possible by reducing the degree of crosslinking. This may be achieved by reducing the genipin concentration in the crosslinking reaction to very low levels (1 mM). This was successful in producing a colored product with various desirable shades of color ranging from tan to brown, or blue, or green. Another method to attenuate the blue color of the Gen-Gel is to treat the Gen-Gel with H₂O₂ to disrupt the chromogenic conjugated systems.

The effect of 3 different H₂O₂ concentrations and 3 different genipin crosslinking concentrations was investigated on product appearance and performance. This experimental matrix is depicted in Table 5.

TABLE 5
Experimental Matrix for H₂O₂ quenched Gen-Gel Synthesis

| | Genipin | Gelatin | Reaction | Quenching |
|---|---|---|---|---|
| Sample i.d. | concentration | concentration | time | solution |
| (lot number) | [mM] | [wt %] | [hrs] | [% H₂O₂] |
| 27786-90B | 5 | 5 | 6 | 15 |
| 27786-90C | 7.5 | 5 | 6 | 15 |
| 27786-90D | 10 | 5 | 6 | 15 |
| 27786-92A | 5 | 5 | 6 | 5 |
| 27786-92B | 7.5 | 5 | 6 | 5 |
| 27786-92C | 10 | 5 | 6 | 5 |
| 27786-96A | 5 | 5 | 6 | 1 |
| 27786-96B | 7.5 | 5 | 6 | 1 |
| 27786-96C | 10 | 5 | 6 | 1 |

General Synthetic Procedure

Genipin was dissolved in de-ionized H₂O to the desired concentration. Un-crosslinked gelatin was added to a concentration of 5% w/v. The resulting suspension was stirred for 6 hrs at RT, over which period a dark blue suspension of genipin-crosslinked gelatin formed in the reaction vessel. The blue suspension was filtered using a Buckner funnel and a Whatman # 54 filter paper. The solid product retained on the filter paper was washed exhaustively with H₂O till the resulting washings had a conductivity reading of <10 μS/cm. The product was re-suspended in a 5% H₂O₂ solution (prepared by diluting a 30 wt % stock solution with de-ionized H₂O). The volume of the 5% H₂O₂ solution was the same as the volume of de-ionized H₂O used in the crosslinking reaction. This reaction vessel was sealed and the reaction stirred for approx. 16-21 hrs at RT. The reaction product was filtered and washed exhaustively with H₂O until no peroxide was detected in the washings, the washings had a pH=7.0 and conductivity reading <10 μS/cm. The filtered H₂O quenched product was transferred to a glass dish and dried at 34° C. for 2 to 4 days. The dried product was ground using a Hamilton-Beach coffee grinder set to the “drip” setting. The ground powder was sized between sieve # 25 and sieve # 80 giving a nominal size range of 177 μm to 710 μm.

Results and Discussion

Variants 27786-90B thru D were quenched with 15% H₂O₂ solution and showed the lightest coloration. Variants 27786-96A thru C were quenched with 1% H₂O₂ solution and had the darkest color. The 27786-92 series was treated with 5% H₂O solution and sits in between the other two in terms of color. Going back to the 27786-90 series—90 B was crosslinked with 5 mM genipin, 90° C. with 7.5 mM genipin, and 90 D with 10 mM genipin. The color accordingly deepend from B to D in this series. Thus the color of the final product was inversely proportional to the concentration H₂O₂ used in quenching and directly proportional to the genipin crosslinking concentration.

After completion of initial performance evaluation, an appreciation for visual appearance of the different H₂O₂ quenched Gen-Gel variants was made. The true physical appearance of the product can only truly be evaluated in its reconstituted form. So the variants detailed in table 5 were reconstituted with thrombin solution and evaluated for physical appearance. FIG. 9 presents a color comparison of the variants from table 5 in their reconstituted form. Upon critical evaluation the 27786-90 and 27786-92 series were acceptable front a visual appearance perspective.

The H₂O₂ quenched Gen-Gel variants were evaluated using the same TEG, % Swell, and EF testing procedures previously described for Gen-Gel. The results are provided in Table 6:

TABLE 6
performance evaluation ofH₂O₂ quenched
Gen-Gel variants from Table 5 (“@ 125%
formulation” = 0.25 g Gen-Gel/ml)

| | Genipin | | | | |
|---|---|---|---|---|---|
| Sample | Concen- | Quenching | % Equi- | EF (lbf) | |
| ID (lot | tration | Solution | librium | @ 125% | TEG |
| number) | (mM) | (% H₂O₂) | Swell | formulation | (A/MA) |
| 27786-90B | 5 | 15 | 794 | 2.7 | 54.9/68.5 |
| 27786-90C | 7.5 | 15 | 722 | 2.7 | 40.1/62.5 |
| 27786-90D | 10 | 15 | 727 | 2.8 | 68.1/69.8 |
| 27786-92A | 5 | 5 | 759 | 3.1 | 71.0/69.1 |
| 27786-92B | 7.5 | 5 | 714 | 3.9 | 75.7/74.0 |
| 27736-92C | 10 | 5 | 729 | 3.5 | 78.2/78.1 |
| 27786-96A | 5 | 1 | 699 | 3.7 | 78.5/79.9 |
| 27786-96B | 7.5 | 1 | 677 | 3.0 | 66.8/70.8 |
| 27786-96C | 10 | 1 | 707 | 4.4 | 60.9/66.1 |

Further results for genipin crosslinking and treatment with H₂O₂ or sodium percarbonate:

Gelatin powder was crosslinked by addition to a 6-10 mM Genipin solution in DIW to create a 5% gelatin suspension. After 4-6 hrs, the excess solution was drained off and solids were captured by a mesh (approximate mesh size No. 270).

The retained solids were re-suspended in a 3%-5% H₂O₂ solution at pH 7 to the approximate reaction volume used during the previous crosslinking step. This solution was allowed to mix overnight for approximately 16-20 hrs. The solution was drained and the solids retained. At least 3 batch rinses or 3 diavolumes of DIW were used to wash the solids, until the conductivity of the solution was <0.1 mS/cm. The solids are then dried in an oven. Crosslinking extent was monitored by measurements of swell where swell is defined as described earlier. Table 7 provides results of the crosslinking and H₂O₂ treatments.

Alternatively, the retained solids can be re-suspended in a 1%-4% sodium percarbonate solution for 1-16 hrs rather than suspension in H₂O₂ solution. Sodium percarbonate was added directly as a powder to the re-suspended crosslinked gelatin solution and consisted of approximately 28% available H₂O₂. The solution was drained and the solids retained. At least 3 batch rinses or 3 diavolumes of DIW were used to wash the solids, until the conductivity of the solution is <0.1 mS/cm. The solids were dried in an oven.

Table 7 provides results of the crosslinking and percarbonate treatments.

TABLE 7
Treatments with H₂O₂

| | Reaction time | | Reaction | |
|---|---|---|---|---|
| Genipin | of gelatin | Peroxide | time with | average |
| concentration | with genipin | concentration | peroxide | Swell |
| (mM) | (hrs) | (% w/w) | (hrs) | (%) |
| 10 | 6 | 5 | 21 | 507 |
| 10 | 6 | 5 | 20 | 625 |
| 10 | 6 | 5 | 21 | 673 |
| 5 | 6 | 3 | 21 | 700 |

TABLE 8
Treatments with sodium percarbonate with genipin at
8 mM and 5 hrs reaction time of gelatin with genipin

| Percarbonate | pH for | Reaction | |
|---|---|---|---|
| concentration | percarbonate | time with | average |
| (% w/w) | treatment | percarbonate (hrs) | Swell (%) |
| 1 | 10 | 3 | 740 |
| 1 | 7 | 16 | 647 |
| 2 | 10 | 1 | 623 |
| 2 | 7 | 3 | 680 |
| 2 | 5 | 16 | 647 |
| 3 | 7 | 1 | 650 |
| 3 | 5 | 3 | 653 |
| 2 | 7 | 1 | 602 |
| 3 | 8 | 2 | 672 |
| 4 | 9 | 3 | 749 |

### Example 5

### H₂O₂ Quenched Gen-Gel—Efficacy in Porcine Liver Model

Experimental Design

Experimental design and objectives axe similar to those described for the in vivo efficacy determination of Gen-Gel described above. In vivo hemostatic efficacy was evaluated in the porcine liver punch-biopsy model described above.

Synthesis of Test Article

10 mM genipin, 6 hrs reaction, 5% H₂O₂ quench [27888-91B (27786-110A)]:

27786-110A was synthesized as per the general synthesis procedure described above. 120 g of un-crosslinked gelatin was used as starting material and all other reagents were used in the ratios described above. After grinding and sizing the end product was sterilized by gamma radiation at a minimum dose of 25 kGy. Post-sterilization the product was re-designated as 27888-91B. The product was packaged in 5 ml “male” syringes (0.8 g/syringe) and submitted for evaluation in the porcine liver model (TEG=good. % Swell=823.1%, EF 0.25 g Gen-Gel/ml=8.43 lbf. color=sage green).

10 mM Genipin, 6 h reaction, Glycine Quench (27888-91A (27888-89A):

27888-80A was synthesized as per the procedure described above. 120 g of uncrosslinked gelatin was used as starting material and all other reagents were used in the ratios described above. After grinding and sizing the end product was sterilized by gamma radiation at a minimum dose of 25 kGy. Post-sterilization the product was re-designated as 27888-91A. The product was packaged in 5 ml “male” syringes (0.8 g/syringe) and submitted for evaluation in the porcine liver model (TEG=good, % Swell=734.6%, EF 0.25 g Gen-Gel/ml=8.24 lbf color=dark blue).

Formulation

In-vivo evaluation of 27888-91A and 27888-91B was performed using the porcine liver punch-biopsy model in 2 animals, one per day. A thrombin/CaCl₂ stock solution was prepared as described above, Test articles 27888-51A were evaluated as their respective formulations (0.25 g Gen-Gel/ml), i.e. 3.2 ml of thrombin solution was used per 0.8 g of Gen-Gel matrix (one syringe). Glu-Gen was used as reference standard. Each sample was rapidly reconstituted 20 times, allowed to rest for 5 minutes, and then re-swooshed 3 more times. 1 ml aliquots of reconstituted test article were dispensed into individual 3 ml volume syringes. These individual 3 ml syringes were then, used to apply test article to liver punch lesions at various time points.

Results and Discussion

The results of the in vivo evaluation of 27888-91A and 2788-91B are presented in FIG. 10. Both test articles equal had hemostatic success rates equal to or better than the Glu-Gel reference standard at all the time points studied. Hemostatic success is defined as no bleeding (FIG. 10a). The performance differential improves in favour of the H₂0₂ quenched variant 27888-91B when the no bleeding or ooze hemostatic success criteria are applied (FIG. 10b).

### Example 6

Gelatin samples were formulated per the package insert for Floseal with a couple key exceptions. First, sodium chloride was used instead of calcium chloride and the gelatin was formulated at 125% solids instead of 100%. The gelatin/thrombin formulations were allowed to stand for 25 minutes and then 1 ml of the preparation was discarded. The other 1 ml of material was applied to the topical hemostasis system (THS). The THS apparatus was previously primed with platelet poor plasma.

The THS is an apparatus designed to simulate a bleeding wound. The artificial wound is a cylindrical hole in a silicone substrate. The surface of the silicone cylinder was coated with a layer of fibrinogen. A syringe pump expelled the clotting fluid (whole blood, plasma, etc.) in this case platelet poor plasma, while the back pressure was recorded, in this experiment the plasma was flowed at a fixed rate of 0.25 ml/min through a small hole at the bottom center of the cylindrical wound. The excess plasma was soaked up with gauze immediately prior to application of the hemostatic matrix. As the plasma continued to flow, 1 ml of the hemostatic matrix was applied to the cylindrical wound. This was immediately covered with wet gauze and a fixed pressure was applied. After 30 seconds the weight was removed and the plasma continued to flow tor 8-10 minutes, at which point the flow was stopped and the clot set aside in a humidity chamber where it stayed for more than 2 hrs. At the end of the two hours, the clot was mounted onto a vibratome at 8° C., where approximately 500 μm thick slabs were sectioned from the clot. These sections were immersed into a PBS buffer. The slabs were stored in a 5° C. refrigerator when not in use. The slab was placed onto a coverslip and imaged with a Nikon A1R confocal microscope running the NIS-Elements Advanced Research v3.22.00 Build 710 software. To collect micrographs, a plan finer 10× objective was used with laser excitation light at 488 nm and an emission collection window from 500-550 nm. A transmitted light image was simultaneously collected using a transmitted light detector. With these imaging parameters, automated stitching performed by the software was used to generate macroscopic maps of samples. Smaller areas of the samples were also characterized by collecting 3D z-stacks of images with an optical slice thickness of 5.125 μm. The composite confocal map was used to identify the gelatin granules that are located at the surface, and which were sectioned. This was important for positioning of the elasticity measurement in the atomic force microscope (AFM). The clot slab was mounted in a Veeco Multimode AFM. The multimode was equipped with a Nanoscope V controller and a JV piezoelectric scanner. The force measurements were made with a Novascan AFM cantiever which supported a 4.5 μm polystyrene sphere. The cantilever's force constant was determined to be 0.779 N/m by the thermal tune method. The cantilever was positioned above the center of the gelatin granule, and then a 16×16 array of force measurements were made. Each force curve involved moving the gelatin granule up into contact with the polystyrene sphere, and continuing to move the granule up until the cantilever deflection reached a preset trigger value of 2 volts, at which point the gelatin was retracted a distance of 1.00 micron horn the trigger location.

Discussion

The fluorescence data shows that the glutaraldehyde crosslinked gelatin is not uniformly crosslinked. Instead, the crosslinking density seems higher around the edges of the granules, with the central portion of the granule being significantly less crosslinked than the edges. In contrast, the genipin crosslinked gelatin appears uniformly (homogeneously) crosslinked throughout the granules. There are no substantial edge effects to the fluorescence intensity. The fluorescence intensity of the genipin and glutaraldehyde crosslinked materials cannot be directly compared, because of the potential fluorescence differences attributed to the crosslinkers themselves. However, the AFM measured elastic modulus measurement show that the genipin crosslinked gelatin is stiffer than the glutaraldehyde crosslinked gelatin, which appears to be softer (more flexible).

### Example 7

### Comparison of Genipin Gelatin Preparation According to the Current Invention and a Genipin Gelatin Preparation According to Chiono et a. 2008—Efficacy in Porcine Liver Model

a) Preparation according to Chiono et al 2008:

Gelatin from bovine skin, type B with a Bloom strength of approx. 75 (Sigma Cat No.G6650-1KG), was dissolved in distilled H₂O at 50° C. to obtain a 10% w/v solution. Genipin (from Wako Cat No.078-03021) was added in order to reach a final concentration of 3.4% w/w. The solution was kept at 50° C. until the solution turned slightly viscous (after approx. 110 min). Thereafter, the solution was poured into a Teflon coated tray (dimension of the tray ˜25.5×33 cm) and left to air-dry at RT for 48 h. The dried films were washed 3 times with distilled H₂O (41 of H₂O per turn) and air dried at RT to reach a constant weight (approx. 69 to 88 h total drying time). The films were ground with a Retsch grinder with a 12 teeth push-fit-rotor and a 0.75 mm ring sieve at 6000 rpm. The granules thus obtained were subjected to gamma sterilization employing a dose range between 25.7-44.2 kGy.

b) Preparation according to present invention:

Gelatin films derived from Type B gelatin with a Bloom strength of approx. 200-400 were ground to a particle size between 0.707 and 0.177 mm. Next, 0.4532 g Genipin (Wako Cat No 078-03021) were dissolved in 400 ml of distilled/de-ionized H₂O and 20.012 g of ground gelatin was added and stirred at RT for 6 hrs. After cross-linking the particles were washed 3 times with 400 ml of de-ionized H₂O and then placed in an oven at 34° C. for 40 hrs to dry. The dried particles were ground with a Retsch grinder with a 12 tooth push-fit-rotor and a 0.75 mm ring sieve at 6000 rpm. The granules thus obtained were subjected to gamma sterilization (minimum dose 25 kGy).

c) Preparation of the Samples in Vivo Performance

Both granules were formulated wife a thrombin solution containing 500 IU/ml thrombin and 40 mM of CaCl₂ in a ratio to obtain a solid to liquid ratio of 17.5% w/w and equilibrated for at least 15 minutes prior to application

As the in vivo performance model, a porcine Liver Model as described in example 3 has been used.

The results that are shown in FIG. 11 demonstrate the superior in vivo performance of the material prepared according to the method of the present invention as compared to the method described in Chiono et al, 2008.

1. A method for producing a hemostatic composition comprising mixing a biocompatible polymer suitable tor use in homeostasis and a genipin-type crosslinker, crosslinking said polymer by said genipin-type crosslinker to obtain a crosslinked biocompatible polymer, and finishing said crosslinked polymer to a pharmaceutically acceptable hemostatic composition.

2. A method for producing a hemostatic composition according to claim 1, wherein the biocompatible polymer suitable for use in hemostasis is present in dry form before the crosslinking step.

3. A method for producing a hemostatic composition according to claim 1, wherein said genipin-type crosslinker is genipin (Methyl (1R,2R,6S)-2-hydroxy-9- (hydroxymethyl)-3-oxabicyclo[4.3.0]nona-4,8-diene-5-carboxylate).

4. A method for producing a hemostatic composition according to claim 1, wherein said biocompatible polymer suitable for use in hemostasis is a protein, a polysaccharide comprising amino groups, a biologic polymer comprising amino groups, a non-biologic polymer comprising amino groups; and derivatives and combinations thereof.

5. A method for producing a hemostatic composition according to claim 1, wherein said biocompatible polymer suitable for use in hemostasis is a protein selected from the group consisting of gelatin, collagen, albumin, hemoglobin, fibrinogen, fibrin, casein, fibronectin, elastin, keratin, and laminim and derivatives and combinations thereof.

6. A method for producing a hemostatic composition according to claim 1, wherein said biocompatible polymer suitable for use in hemostasis is a polysaccharide comprising amino groups selected from the group consisting of glycosaminoglycans, pectins, modified starch comprising amino groups, modified cellulose comprising amino groups, modified dextran comprising amino groups, modified hemicellulose comprising amino groups, modified xylan comprising amino groups, modified agarose comprising amino groups, modified alginate comprising amino groups, chain and chitosan; and derivatives and combinations thereof.

7. A method for producing a hemostatic composition according to claim 1, wherein said biocompatible polymer suitable for use in hemostasis is a polymer selected from the group consisting of polyacrylamides, polymethacrylamides, polyethyleneimines, polylysine, polyarginine and polyamidoamine (PAMAM) dendrimers.

8. A method for producing a hemostatic composition according to claim 1, wherein said crosslinked biocompatible polymer is subjected to air oxidation step, preferably to a treatment with sodium percarbonate, sodium hypochlorite, chlorine water or H₂O₂, especially to a treatment with a 1 to 15% H₂O₂ solution.

9. A method for producing a hemostatic composition according to claim 1, wherein the crosslinking step is performed in aqueous solution, preferably in a PBS/ethanol buffer, especially at a pH of 7 to 8, or in de-ionized water.

10. A method for producing a hemostatic composition according to claim. 1, wherein the crosslinking step is performed in an aqueous buffer with a pH of 4 to 12 containing up to 50% v/v) of water-miscible organic solvent and/or one or more processing aids.

11. A method for producing a hemostatic composition according to claim 1, wherein the crosslinking step is performed at a temperature of 4 to 45° C., preferably of 15 to 45° C., especially of 20 to 40° C.

12. A method for producing a hemostatic composition according to claim 1, wherein the crosslinking step is followed by a quenching step, especially with an amino-group containing quencher, preferably an amino acid, especially glycine.

13. A method for producing a hemostatic composition according to claim 1, wherein the crosslinking step is followed by raising the pH to 8 to 14, by addition of nucleophiles or by raising the pH to 8 to 14 and addition of nucleophiles.

14. A method for producing a hemostatic composition according to claim 1, wherein the crosslinked biocompatible polymer is washed after the crosslinking step, preferably with methanol, ethanol or water, especially by de-ionized water.

15. A method tor producing a hemostatic composition according to claim 1, wherein the crosslinked biocompatible polymer is washed after the crosslinking step with an aqueous buffer containing up to 50% (v/v) of water-miscible organic solvent and/or one or more processing aids.

16. A method for producing a hemostatic composition according to claim 1, wherein said crosslinked biocompatible polymer is dried or provided as a wet hydrogel in suitable biocompatible buffer.

17. A method tor producing a hemostatic composition according to claim 1, wherein said crosslinked biocompatible polymer is dried to have a moisture of below 15%, preferably below 10%, more preferred below 5%, especially below 1%.

18. A method for producing a hemostatic composition according to claim 1, wherein the biocompatible polymer suitable for use in hemostasis is a gelatin with a Bloom strength of 200 to 400, especially a type B gelatin with a Bloom strength of 200 to 400.

19. Hemostatic composition comprising a crosslinked biocompatible polymer obtainable by a method according to claim 1.

20. Hemostatic composition according to claim 19, wherein the crosslinked biocompatible polymer is a gelatin polymer.

21. Hemostatic composition according to claim 19, wherein the crosslinked biocompatible polymer is a type B gelatin polymer.

22. Hemostatic composition according to claim 19, wherein the crosslinked biocompatible polymer is present in particulate form, preferably as granular material.

23. Hemostatic composition according to 19, wherein the crosslinked biocompatible polymer is present in dry form.

24. Hemostatic composition according to claim 19, wherein the crosslinked biocompatible polymer is a gelatin with a Bloom strength of 200 to 400.

25. Hemostatic composition according to claim 19, wherein the crosslinked biocompatible polymer has an equilibrium swell of at least 400%, preferably of 600 to 900%.

26. Hemostatic composition to claim 19 for use in the treatment of an injury selected from the group consisting of a wound, a hemorrhage, damaged tissue, bleeding tissue and/or bone defects.

27. Method of treating an injury selected from the group consisting of a wound, a hemorrhage, damaged tissue and/or bleeding tissue comprising administering a hemostatic composition according to claim 19 to the site of injury.

28. Kit for the treatment of an injury selected from the group consisting of a wound, a hemorrhage, damaged tissue and/or bleeding tissue comprising
a) a hemostatic composition according to claim 19; and
b) instructions for use.

29. Kit according to claim 28, the hemostatic composition is in dry form, the kit further comprising a pharmaceutically acceptable diluent for reconstitution of the hemostatic composition.

30. Kit according to claim 28, further comprising a pharmaceutically acceptable diluent containing a substance selected from the group consisting of NaCl, CaCl₂, sodium acetate and mannitol.

31. Kit according to claim 28, wherein the pharmaceutically acceptable diluent comprises a buffer or buffer system, preferably at a pH of 3.0 to 10.0.

32. Kit according to claim 28, wherein the pharmaceutically acceptable diluent comprises thrombin, preferably 10 to 1000 I.U. thrombin/ml, especially 250 to 700 I.U. thrombin/ml.

33. Method for providing a ready to use form of a hemostatic composition according to claim 19, wherein the hemostatic composition is provided in a first syringe and a pharmaceutically acceptable diluent for reconstitution is provided in a second syringe, the first and the second syringe are connected to each other, and the fluid is brought into the first syringe to produce a flowable form of the hemostatic composition; and optionally returning the flowable form of the hemostatic composition to the second syringe at least once.

34. Method according to claim 33 wherein the flowable form of the hemostatic composition contains particles of which more than 50% (w/w) have a size of 100 to 1000 μm, preferably particles of which more than 80% (w/w) have a size of 100 to 1000 μm.

35. Method according to claim 33, wherein the pharmaceutically acceptable diluent further comprises thrombin, preferably 10 to 1000 I.U. thrombin/ml, especially 50 to 500 I.U. thrombin/ml.

36. Method according to claim 33 wherein the crosslinked biocompatible polymer is crosslinked gelatin, especially crosslinked type B gelatin.

37. Ready to use hemostatic composition comprising a crosslinked biocompatible polymer obtainable by a method according to claim 33.

38. Ready to use hemostatic composition according to claim 37 wherein the flowable form contains crosslinked biocompatible polymer in an amount of 5 to 30 % (w/w), preferably of 10 to 25% (w/w), especially of 12 to 20% (w/w).
